(19) United States
(12) Patent Application Publication
(10) Pub. No.: US 2025/0145961 A1
(43) Pub. Date: May 8, 2025

(54) GENERATING BONE MARROW ORGANOIDS

(71) Applicant: The University of Birmingham, Birmingham, West Midlands (GB)

(72) Inventors: Abdulla KHAN, Birmingham (GB); Bethan Psaila, Birmingham (GB)

(21) Appl. No.: 18/838,225

(22) PCT Filed: Feb. 15, 2023
(86) PCT No.: PCT/GB2023/050348

§ 371 Date: Aug. 13, 2024

(30) Foreign Application Priority Data
Feb. 15, 2022 (GB) 2202025.9
Nov. 8, 2022 (GB) 2216647.4

(51) Int. Cl. C12N 5/077; C12N 5/0789

(52) U.S. Cl. CPC C12N 5/0669; C12N 5/0647; C12N 2501/115; C12N 2501/125; C12N 2501/155; C12N 2501/165; C12N 2501/22; C12N 2501/2303; C12N 2501/2306; C12N 2501/26; C12N 2501/91; C12N 2503/04; C12N 2506/45

(57) ABSTRACT

The invention relates to a method of generating bone marrow organoids from pluripotent stem cells wherein said method comprises: a) driving induced pluripotent stem cells (iPSCs) to form mesodermal aggregates; b) inducing vascular and hematopoietic commitment of the mesodermal aggregates by further culturing the mesodermal aggregates in medium, supplemented with recombinant BMP4, FGF2, VEGFA and SCF; c) embedding the mesodermal aggregates in a hydrogel, and incubating the hydrogel in a sprouting medium comprising: a media suitable for stem cell maintenance supplemented with cytokines for differentiation, to form bone marrow organoids; and optionally d) separating the bone marrow organoids from the hydrogel for further free culture.

The present invention relates to a method of generating bone marrow organoids from pluripotent stem cells, a model for bone marrow disorders including blood malignancies and uses for screening for efficacy of pharmacological and genetic manipulations for blood cancer treatment.

### BACKGROUND

The bone and bone marrow are highly complex organs rich in multiple cell lineages, and are responsible for the generation and maintenance of blood cells. Current in vitro models of the bone and bone marrow lack both the architecture and cellular diversity to effectively model these systems and are typically composed of chips containing individual cell lineages obtained from primary human or mouse tissues or differentiated from stem cells. 3D cell culture allows for the development of a system that more accurately mimics the cellular complexity of native bone marrow. Mouse models are also often used to model human hematopoiesis. However, mouse bone marrow has certain differences to the human equivalent, and for many blood cancer types, the available mouse models fail to accurately reproduce the human pathologies. This limits their application for the study of mechanisms of disease and the identification and validation of new therapies.

A physiologically accurate bone model is therefore needed. While substantial advances have been made in the treatment of some blood cancers, many blood cancer remain incurable and there is a huge unmet need for new biological systems that may enable improved disease modelling and target screening. This is particularly true for bone marrow fibrosis, for example that occurring in a bone marrow malignancy called myelofibrosis, where a cancer clone drives excessive deposition of reticulin fibrosis resulting in scarring and destruction of the haematopoietic tissue, bone marrow failure, and typically death within 5-10 years from diagnosis. In addition, it is not currently possible to keep cells isolated from patients with certain types of blood cancer alive for sufficient time following sampling for mechanistic study or pharmacological or genetic manipulation, as they are poorly viable following sampling once they are not supported by the bone marrow niche.

Existing bone marrow models generally include minimal cell lineages or component parts, for example the co-culture of endothelial cells with immune cell subtypes in channels or microfluidic chips. However, this approach is limited in that it does not include the many specialist lineages of the bone marrow, or its unique architectural tissue organisation.

Therefore, there is a clear need for an improved model for bone marrow diseases, and in particular blood cancers—for example, to understand how the bone marrow stroma supports production of blood from healthy haematopoietic stem cells, to study the interactions between the malignant haematopoietic cells and bone marrow stroma and to identify and validate new targets for therapy.

The bone marrow organoids described here provide a transformative solution for modelling bone marrow function, blood production and blood cancers as well as target screening.

### STATEMENT OF INVENTION

According to a first aspect of the present invention, provided herein is a method of generating bone marrow organoids from pluripotent stem cells wherein said method comprises:
a. driving induced pluripotent stem cells (iPSCs) to form mesodermal aggregates;
b. inducing vascular and haematopoietic commitment of the mesodermal aggregates by further culturing the mesodermal aggregates in medium, supplemented with recombinant BMP4, FGF2, VEGFA, Flt-3 and SCF;
c. embedding the mesodermal aggregates in a hydrogel, and incubating the hydrogel in a sprouting medium comprising: a media suitable for stem cell maintenance supplemented with cytokines for differentiation, to form bone marrow organoids; and optionally
d. separating the bone marrow organoids from the hydrogel for further free culture.

Advantageously, the methods of the present invention lead to the generation of a physiologically accurate bone marrow niche that replicates the cellular, molecular and architectural features of hematopoietic tissues including key specialist lineages of the bone marrow. A physiologically accurate bone marrow will facilitate effective studies into bone marrow biology, bone marrow cancers, and for the development of novel therapeutics.

### Step a. Driving Induced Pluripotent Stem Cells (iPSCs) to Form Mesodermal Aggregates

The skilled person will be familiar with routine methods to form mesodermal aggregates from iPSCs. In one embodiment, the mesodermal aggregates are formed by:
i. incubating induced pluripotent stem cells (iPSCs) to induce the formation of iPSC aggregates; and
ii. culturing the iPSC aggregates in a mesoderm-inducing medium comprising a media for stem cell maintenance supplemented with BMP4, FGF2, and VEGFA, to induce formation of mesodermal aggregates.

Alternative methods of mesoderm induction are known to the skilled person, for example culturing iPSCs in low oxygen (e.g. 1-5%) with BMP4, with or without a WNT inhibitor, such as CHIR99021.

The skilled person will recognise that the term “mesodermal aggregates” may also be referred to as “embryoid bodies (EBs)”.

In one embodiment, the induced pluripotent stem cells are human iPSCs (hiPSCs). The provided iPSCs, such as hiPSCs, may be undifferentiated.

Prior to the induction of iPSC aggregates, the iPSCs may be maintained and cultured on a basement/basal matrix (such as hESC-qualified Matrigel™) in mTeSR1 medium. Alternatively, the iPSCs may be cultured on other gel substrates such as, Geltrex™ (LDEV-Free Reduced Growth Factor Basement Membrane Matrix, which is a soluble form of basement membrane extracted from murine Engelbreth-Holm-Swarm (EHS) tumors), vitronectin or laminin cell culture matrix (Biolamina™) and maintained in other stem cell differentiation culture media such as StemFlex™ or E8 media, or equivalents thereof. The iPSCs may be cultured on tissue culture plates coated with the collagen matrix or other gel substrate.

In one embodiment, the iPSCs are passaged one or more times, for example at about 20% or more confluence. In one embodiment, the iPSCs are passaged one or more times, for example at about 50% or more confluence. In another embodiment, the iPSCs are passaged one or more times, for example at about 20-90% confluence. Preferably, the iPSCs may be passaged one or more times, for example at about 70-80% confluence. The iPSCs may be detached for passaging and/or aggregate formation, for example using physical/mechanical detachment and/or non-physical/mechanical detachment, such as using EDTA or enzymatic detachment. Preferably EDTA detachment is used together with mechanical dissociation, for example by pipetting action. In another embodiment, iPSCs may be detached using a method which generates a single cell suspension, for example using TRYPLE or Accutase.

For iPSC aggregate formation, detached iPSCs may be cultured in stem cell differentiation medium for a period of at least 8 hours. In another embodiment, the detached iPSCs may be cultured in stem cell differentiation medium for a period of at least 12 hours. In another embodiment, the detached iPSCs may be cultured in stem cell differentiation medium for a period of about 8-24 hours.

The stem cell differentiation medium may comprise basal media supplemented with a ROCK inhibitor, such as Y-27632. In one embodiment, the stem cell differentiation media comprises or consists of StemFlex™ (or equivalents thereof) optionally supplemented with ROCK inhibitor supplements, such as RevitaCell™ (Thermo), or equivalents thereof.

In one embodiment, the iPSCs for aggregate formation are cultured on ultra-low adhesion plates, for example comprising a covalently bound hydrogel layer that inhibits cellular attachment.

Following iPSC aggregate detachment, the iPSC aggregates may be incubated for a period before adding them to mesoderm induction medium. The incubation may be for a period of 8-24 hours, preferably about 8-12 hours. The incubation may be under standard cell maintenance conditions, such as 5% CO₂ at 37° C. The resulting iPSC aggregates may be collected by gravitation or centrifugation and resuspended in the mesoderm-inducing medium (phase I medium) of step b. In one embodiment, the day iPSC aggregates are transferred for culture mesoderm-inducing medium is day 0. Culturing the iPSC aggregates in the mesoderm-inducing medium may be under suitable cell growth conditions, for example at 5% O₂, 5% CO₂, and 37° C.

The early mesoderm induction step may comprise incubation of the iPSC aggregates over a period of time sufficient to form mesoderm. Culturing the iPSC aggregates in the mesoderm-inducing medium may be for a period of about 72 hours. In another embodiment, culturing the iPSC aggregates in the mesoderm-inducing medium may be for a period of about 3 days. In another embodiment, culturing the iPSC aggregates in the mesoderm-inducing medium may be for a period of about 2-5 days. In another embodiment, culturing the iPSC aggregates in the mesoderm-inducing medium may be for a period of about 3-5 days. In another embodiment, culturing the iPSC aggregates in the mesoderm-inducing medium may be for a period of about 3-7 days. In another embodiment, culturing the iPSC aggregates in the mesoderm-inducing medium may be for a period of between about 60 hours and about 90 hours.

Additionally, or alternatively, culturing the iPSC aggregates in the mesoderm-inducing medium may be for a period until aggregates reached an average size of about 200-250 μm. The size of the aggregate is understood to be the average of the largest diameter of the iPSC aggregates.

The mesoderm may be considered formed (i.e. for moving onto the next step) when at least 60% of the cells are mesodermal cells. In another embodiment, the mesoderm may be considered formed when at least 70% of the cells are early mesodermal cells. In another embodiment, the mesoderm may be considered formed when at least 80% of the cells are mesodermal cells. The skilled person will understand that the formation of mesoderm cells may be provided as much as necessary, with the understanding that the number or percentage of mesoderm cells formed at this stage will have an impact on the end yield of bone marrow organoids. In particular, the higher number of aggregates will give a higher number of bone marrow organoids, but a higher number of mesodermal cells will give a more efficient differentiation into bone marrow lineages. Early mesodermal cells or tissue may be identified by the expression of key marker genes, such as one or more, or all of, Brachyury, Snail, TBX6 and N-cadherin, which then subsequently mature into definitive mesoderm. Therefore, in one embodiment, the production of mesodermal tissue from the iPSC aggregates may be identified by detecting the presence of mesodermal markers using any method known in the art. The mesodermal markers may comprise or consist of one or more, or all, of Brachyury, Snail, TBX6 and N-cadherin. The cell marker may be detected by protein expression, for example using immunofluorescence or mRNA expression (e.g. using qRT-PCR).

The mesoderm-inducing medium may be a chemically defined medium (CDM). In one embodiment, the mesoderm-inducing medium comprises a basal medium, preferably a stem cell differentiation culture media. In one embodiment the mesoderm-inducing medium comprises a stem cell differentiation media, such as APEL2 or StemPro, or equivalents thereof. The mesoderm-inducing medium may comprise a ROCK inhibitor, such as Y-27632.

The BMP4 may be provided in the mesoderm-inducing medium at a concentration of between about 10 and about 100 ng/ml. In another embodiment, the BMP4 may be provided in the mesoderm-inducing medium at a concentration of between about 40 and about 60 ng/ml. In a preferred embodiment, the BMP4 is provided in the mesoderm-inducing medium at a concentration of 50 ng/ml.

The FGF2 may be provided in the mesoderm-inducing medium at a concentration of between about 10 and about 50 ng/ml. In a preferred embodiment, the FGF2 is provided in the mesoderm-inducing medium at a concentration of 50 ng/ml. In one embodiment, the medium comprises FGF2 in an amount suitable to promote mesoderm differentiation and priming for early lineage fibroblast.

The VEGFA may be provided in the mesoderm-inducing medium at a concentration of between about 10 and about 50 ng/ml. In a preferred embodiment, the VEGFA is provided in the mesoderm-inducing medium at a concentration of 50 ng/ml. In one embodiment, the medium comprises VEGFA in an amount suitable to promote mesoderm commitment and prime early endothelial/haematopoeitic bipotent progenitors.

In one embodiment, BMP4 is used at a concentration of 50 ng/ml, FGF2 is used at a concentration of 50 ng/ml, and VEGFA is used at a concentration of 50 ng/ml.

### Step b. Inducing Vascular and Haematopoietic Commitment

The step of inducing vascular and haematopoietic commitment may comprise culturing the mesodermal aggregates from step a. in mesoderm-inducing medium (Phase II media) comprising, BMP4, FGF2, VEGFA, Flt-3 and SCF. Preferably, the medium is replaced between steps a and b, for example by harvesting the mesodermal aggregates by gravitation or centrifugation and resuspension in the mesoderm-inducing medium. In an alternative embodiment, the media may not be replaced, but is may be further supplemented with the required cytokines, such as BMP4, FGF2, VEGFA, Flt-3 and SCF.

The medium may be capable of inducing mesoderm. The medium may be a chemically defined medium (CDM). In one embodiment, the medium comprises a basal medium, preferably a stem cell differentiation culture media. In one embodiment the medium comprises stem cell differentiation medium, such as APEL2™ (as described by Ng et al (Nat Protoc. 2008; 3(5):768-76. doi: 10.1038/nprot.2008.42), which is herein incorporated by reference.

The BMP4 may be provided in the medium at a concentration of between about 10 and about 100 ng/ml. In another embodiment, the BMP4 may be provided in the medium at a concentration of between about 40 and about 60 ng/ml. In a preferred embodiment, the BMP4 is provided in the medium at a concentration of 50 ng/ml.

The FGF2 may be provided in the medium at a concentration of between about 10 and about 50 ng/ml. In a preferred embodiment, the FGF2 is provided in the medium at a concentration of 50 ng/ml. In one embodiment, the medium comprises FGF2 in an amount suitable to promote mesoderm differentiation and priming for early lineage fibroblast.

The VEGFA may be provided in the medium at a concentration of between about 10 and about 50 ng/ml. In a preferred embodiment, the VEGFA is provided in the medium at a concentration of 50 ng/ml. In one embodiment, the medium comprises VEGFA in an amount suitable to promote mesoderm commitment and prime early endothelial/haematopoeitic bipotent progenitors.

The Flt-3 may be provided in the medium at a concentration of between about 10 and about 100 ng/ml. In another embodiment, the Flt-3 may be provided in the medium at a concentration of between about 10 and about 40 ng/ml. In a preferred embodiment, Flt-3 is provided in the medium at a concentration of 25 ng/ml. In one embodiment, the medium comprises Flt-3 in an amount suitable to promote haematopoeitic commitment.

The SCF (stem cell factor) may be provided in the medium at a concentration of about 1-100 ng/mL. In another embodiment, the SCF is provided in the medium at a concentration of about 10-30 ng/mL. In one embodiment, the SCF is provided in the medium at a concentration of 25 ng/ml. In one embodiment, the medium comprises SCF in an amount suitable to promote haematopoeisis (generation of haematopoeitic lineages).

In one embodiment, BMP4 is used at a concentration of 50 ng/ml, FGF2 is used at a concentration of 50 ng/ml, VEGFA is used at a concentration of 50 ng/ml, SCF is used at a concentration of 25 ng/ml and Flt3 is used at a concentration of 25 ng/ml.

The medium may be further supplemented with one or more additional cytokines, such as IL7 or other interleukins to induce lymphopoeisis. Additionally or alternatively, the additional cytokines may include one or more of IL11, FLT3L, GM-CSF, IL1, IL12, IL13, IL33, TPO, IL3, IL6, IL2, IL10 and IL8.

The vascular and haematopoietic commitment step may comprise culturing the mesodermal aggregates for a period of between about 36 and 72 hours, preferably for a period of about 40-60 hours, more preferably for about 48 hours, or until the mesodermal aggregates achieve an average size of about 350-400 um (which may be about d4-6, preferably d5). Additionally, or alternatively, the vascular and haematopoietic commitment step may comprise culturing the mesodermal aggregates for a period until there are markers of early endothelial and haematopoeitic differentiation. Such markers may include one or more, or all of CD144, CD31, RUNX1, and GATA1. Markers may be detected by any suitable means, such as by qRT-PCR.

Following vascular and haematopoietic commitment, the mesodermal aggregates may be collected, for example via centrifugation, prior to embedding in the hydrogel (step c).

The culture conditions of step b may be standard cell culture conditions, such as 5% O₂, 5% CO₂, and 37° C. The culture conditions of step b may be normoxic conditions (e.g. about 20-21% O₂), or with 5% O₂.

### Step c. Embed in Hydrogel (Sprouting Stage)

The media for stem cell maintenance of step c. may comprise stem cell differentiation medium, such as APEL2™ (as described by Ng et al (Nat Protoc. 2008; 3(5):768-76. doi: 10.1038/nprot.2008.42), which is herein incorporated by reference, or equivalents thereof.

In one embodiment, the sprouting medium comprises a cytokine and/or growth factor cocktail for generating the desired mix of haematopoietic cell lineages. In one embodiment, the sprouting medium comprises a media for stem cell maintenance supplemented with growthfactors, for example selected from VEGFA, VEGFC, FGF2, SCF, Flt3, IL-3, IL-6, TPO, EPO, G-CSF, FBS and heparin, or combinations thereof. In one embodiment, the sprouting medium comprises a media for stem cell maintenance supplemented with VEGFA, VEGFC, FGF2, SCF, Flt3, IL-3, IL-6, TPO, EPO, and G-CSF, and optionally FBS and/or heparin, to form bone marrow organoids. In another embodiment, the sprouting medium comprises a media for stem cell maintenance supplemented with VEGFA, FGF2, SCF, Flt3, IL-3, IL-6, TPO, EPO, and G-CSF, and optionally FBS and/or heparin, to form bone marrow organoids. In another embodiment, the sprouting medium comprises a media for stem cell maintenance supplemented with VEGF, FGF2, TPO, SCF, EPO, G-CSF. In another embodiment, the sprouting medium comprises a media for stem cell maintenance supplemented with VEGFA, VEGFC, FGF2, SCF, Flt3, TPO, EPO, and G-CSF, and optionally FBS and/or heparin, to form bone marrow organoids. In one embodiment, the sprouting medium comprises a media for stem cell maintenance supplemented with VEGFA, VEGFC, FGF2, SCF, Flt3, IL-3, IL-6, EPO, and G-CSF, and optionally FBS and/or heparin, to form bone marrow organoids. In another embodiment, the sprouting medium comprises a media for stem cell maintenance supplemented with VEGFA, FGF2, SCF, Flt3, IL-3, IL-6, EPO, and G-CSF, and optionally FBS and/or heparin, to form bone marrow organoids. In another embodiment, the sprouting medium comprises a media for stem cell maintenance supplemented with VEGF, FGF2, SCF, EPO, G-CSF. In another embodiment, the sprouting medium comprises a media for stem cell maintenance supplemented with VEGFA, VEGFC, FGF2, SCF, Flt3, EPO, and G-CSF, and optionally FBS and/or heparin, to form bone marrow organoids. The media may additionally or alternatively comprise IL3, IL6, IL2, IL8, IL10, IL7 other fibroblast growth factors and VEGFs. In another embodiment, sprouting medium comprises a media for stem cell maintenance supplemented with VEGF, FGF2, TPO, SCF, Flt3 and optionally one or more of EPO, G-CSF, M-CSF, GM-CSF, IL3, IL6, and IL7, and optionally FBS and/or heparin, to form bone marrow organoids. Any sprouting medium disclosed herein may comprise one or more small molecule (e.g. less than 900 Da) enhancers of hematopoietic or vascular differentiation. Enhancers of hematopoietic or vascular differentiation may comprise one or more of Forskolin, UM171 and SR1.

The VEGFA in the sprouting medium may be human VEGFA. In one embodiment VEGFA is present in the sprouting medium at a concentration of at least 2 ng/ml. In another embodiment, VEGFA is present in the sprouting medium at a concentration of between about 2 and 100 ng/ml. In one embodiment, VEGFA is present in the sprouting medium at a concentration of between about 5 and 100 ng/ml. In another embodiment, VEGFA is present in the sprouting medium at a concentration of between about 5 and 50 ng/ml. In another embodiment, VEGFA is present in the sprouting medium at a concentration of between about 40 and 60 ng/ml. In a preferred embodiment, VEGFA is present in the sprouting medium at a concentration of about 50 ng/ml. In one embodiment, the sprouting medium comprises VEGFA in an amount suitable to promote vascular commitment and endothelial sprouting.

The FGF2 in the sprouting medium may be human FGF2. In one embodiment FGF2 is present in the sprouting medium at a concentration of at least about 1 ng/ml. In another embodiment FGF2 is present in the sprouting medium at a concentration of 1-100 ng/ml. In another embodiment FGF2 is present in the sprouting medium at a concentration of 1-500 ng/ml. In one embodiment FGF2 is present in the sprouting medium at a concentration of 10-100 ng/ml. In another embodiment FGF2 is present in the sprouting medium at a concentration of 10-500 ng/ml. In one embodiment, the sprouting medium comprises FGF2 in an amount suitable to promote growth of fibroblast, mesenchymal stromal cells, and support vasculogenesis.

The sprouting medium may comprise between about 1 and 20% Foetal Bovine Serum (FBS). In one embodiment, the sprouting medium comprises at least about 1% Foetal Bovine Serum (FBS). In a preferred embodiment, the sprouting medium comprises about 5% Foetal Bovine Serum (FBS). In one embodiment, the sprouting medium comprises FBS in an amount suitable to promote endothelial sprouting and vasculogenesis. The FBS may be substituted with knock-out serum.

The sprouting medium may comprise at least about 1 U/mL heparin sulfate. The sprouting medium may comprise about 1-100 U/mL heparin sulfate. The sprouting medium may comprise about 1-500 U/mL heparin sulfate. In another embodiment, the sprouting medium may comprise about 2-10 U/mL heparin sulfate. The sprouting medium may comprise about 2-500 U/mL heparin sulfate. In one embodiment, the sprouting medium comprises at least about 5 U/mL heparin sulfate. In one embodiment, the sprouting medium comprises heparin sulfate in an amount suitable to promote FGF signalling, and optionally proplatelet formation from megakaryocytes.

The sprouting medium may comprise at least about 1 ng/ml Interleukin 3 (IL3). The sprouting medium may comprise about 1-50 ng/ml Interleukin 3 (IL3). In one embodiment, the sprouting medium comprises at least about 10 ng/ml Interleukin 3 (IL3). In one embodiment, the sprouting medium comprises about 1-100 ng/ml Interleukin 3 (IL3). In one embodiment, the sprouting medium comprises about 1-500 ng/ml Interleukin 3 (IL3). In one embodiment, the sprouting medium comprises IL3 in an amount suitable to promote haematopoeitic cell development and haematopoeitic commitment.

The sprouting medium may comprise at least about 1 ng/ml Interleukin 6 (IL6). The sprouting medium may comprise about 1-50 ng/ml Interleukin 6 (IL6). In another embodiment, the sprouting medium may comprise at least about 10 ng/ml Interleukin 6 (IL6). The sprouting medium may comprise about 1-100 ng/ml Interleukin 6 (IL6). The sprouting medium may comprise about 1-500 ng/ml Interleukin 6 (IL6). In one embodiment, the sprouting medium comprises IL6 in an amount suitable to promote haematopoeitic cell development and haematopoeitic commitment.

The sprouting medium may comprise at least about 1 ng/ml SCF The sprouting medium may comprise about 1-100 ng/ml SCF. In another embodiment, the sprouting medium may comprise about 1-500 ng/ml SCF. In another embodiment, the sprouting medium may comprise about 10-30 ng/ml SCF. In another embodiment, the sprouting medium may comprise about 10-500 ng/ml SCF. In another embodiment, the sprouting medium may comprise at least about 25 ng/ml SCF. In one embodiment, the sprouting medium comprises SCF in an amount suitable to promote haematopoeitic cell development and haematopoeitic commitment.

The sprouting medium may comprise at least about 1 ng/ml Flt3. The sprouting medium may comprise about 1-100 ng/ml Flt3. The sprouting medium may comprise about 1-500 ng/ml Flt3. In another embodiment, the sprouting medium may comprise about 10-30 ng/ml Flt3. The sprouting medium may comprise about 10-500 ng/ml Flt3. In another embodiment, the sprouting medium may comprise at least about 25 ng/ml Flt3. In one embodiment, the sprouting medium comprises Flt3 in an amount suitable to promote haematopoeitic cell development and haematopoeitic commitment.

The sprouting medium may comprise at least about 1 ng/ml thrombopoietin (TPO). The sprouting medium may comprise about 1-50 ng/ml thrombopoietin (TPO). The sprouting medium may comprise about 1-500 ng/ml thrombopoietin (TPO). In another embodiment, the sprouting medium may comprise about 10-30 ng/ml thrombopoietin (TPO). The sprouting medium may comprise about 10-500 ng/ml thrombopoietin (TPO). In another embodiment, the sprouting medium may comprise at least about 25 ng/ml thrombopoietin (TPO). In one embodiment, the sprouting medium comprises TPO in an amount suitable to promote haematopeoisis and the development of megakaryocytes (megakaryo- and thrombopoiesis).

The sprouting medium may comprise at least about 1 ng/ml Erythropoietin (EPO). The sprouting medium may comprise about 1-100 ng/ml Erythropoietin (EPO). The sprouting medium may comprise about 1-500 ng/ml Erythropoietin (EPO). In another embodiment, the sprouting medium may comprise about 10-30 ng/ml Erythropoietin (EPO). The sprouting medium may comprise about 10-500 ng/ml Erythropoietin (EPO). In another embodiment, the sprouting medium may comprise at least about 25 ng/ml Erythropoietin (EPO). In one embodiment, the sprouting medium comprises EPO in an amount suitable to promote development of erythroid cells (erythropoiesis).

The sprouting medium may comprise at least about 1 ng/ml Granulocyte Colony Stimulating Factor (G-CSF). The sprouting medium may comprise about 1-100 ng/ml Granulocyte Colony Stimulating Factor (G-CSF). The sprouting medium may comprise about 1-500 ng/ml Granulocyte Colony Stimulating Factor (G-CSF). In another embodiment, the sprouting medium may comprise about 10-30 ng/ml Granulocyte Colony Stimulating Factor (G-CSF). The sprouting medium may comprise about 10-500 ng/ml Granulocyte Colony Stimulating Factor (G-CSF). In another embodiment, the sprouting medium may comprise at least about 25 ng/ml Granulocyte Colony Stimulating Factor (G-CSF). In one embodiment, the sprouting medium comprises G-CSF in an amount suitable to promote commitment to neutrophil/monocyte lineage (development of this arm of immune cells).

The sprouting medium may comprise about 1-100 ng/ml BMP4. The sprouting medium may comprise about 1-500 ng/ml BMP4. In another embodiment, the sprouting medium may comprise about 10-30 ng/ml BMP4. The sprouting medium may comprise about 10-500 ng/ml BMP4. In another embodiment, the sprouting medium may comprise at least about 25 ng/mL BMP4. In one embodiment, the sprouting medium comprises BMP4 in an amount suitable to promote haematopoeisis.

In one embodiment, the embedded mesoderm aggregates are maintained until about d12. In one embodiment, the embedded mesoderm aggregates are maintained in sprouting medium for at about 1 week. In another embodiment, the embedded mesoderm aggregates are maintained in the sprouting medium for at about 5-10 days, preferably about 7 days.

The sprouting medium may be changed during the maintenance period, for example the sprouting medium may be changed every 48-84 hours, preferably every 72 hours

Sprouting medium changes may comprise 100% fresh medium or may comprise a mixture of fresh and conditioned sprouting medium. A mixture of fresh and conditioned sprouting medium may be used at d5 onwards. The fresh and conditioned sprouting medium may comprise between 20:80 and 80:20 fresh:conditioned media. In one embodiment, the fresh and conditioned sprouting medium may comprise about 60:40 fresh:conditioned media.

The conditioned media can provide a benefit as differentiating cells are secreting factors (cytokines and matrix components) which can help the niche develop, as well as support haematopoiesis.

The skilled person may adjust the concentration and/or composition of cytokines in order to alter the composition of the resulting bone marrow organoid. For example, part way (e.g. about half way) through the sprouting phase the concentration of EPO may be increased to produce more erythrocytes, or EPO may be reduced along with an increase in the SCF/FLT3 content to produce more HSPCs. The increase or decrease may be sufficient to allow the production of a desired cell composition in the bone marrow organoids. The decrease of a given cytokine may be about a 10%, 30%, 50%, 80%, or 90% decrease. The increase of a given cytokine may be about a 10%, 30%, 50%, 80%, 100%, 150% or 200% increase.

In one embodiment, the sprouting medium may further comprise VEGFC, for example for developing sinusoid specific endothelial vasculature. In an embodiment wherein VEGFC is provided into the sprouting medium the VEGFC may be added at a later period, for example from d3 (from the overall differentiation timeline). The sprouting medium may comprise VEGFC at a concentration of about 10-100 ng/ml. In another embodiment, the sprouting medium may comprise VEGFC at a concentration of about 20-30 ng/ml. In a preferred embodiment, the sprouting medium may comprise VEGFC at a concentration of about 25 ng/ml.

The hydrogel may be a mixed matrix hydrogel. In one embodiment, the mixed matrix hydrogel comprises collagen I and collagen IV. The ratio of collagen I to collagen IV may be 1:1 to 3:1. In one embodiment, the ratio of collagen I to collagen IV is 1:1. In one embodiment the hydrogel comprises or consists of collagen I or collagen IV.

The collagen may be provided at a concentration of between about 0.1 and 3.5 mg/ml. The collagen may be provided at a concentration of between about 1 and 3.5 mg/ml. In one embodiment, the collagen is provided at a concentration of about 1 mg/ml. In one embodiment, the mixed matrix hydrogel comprises Matrigel.

In one embodiment, the hydrogel comprises a natural polymer, such as collagen or fibrin. In another embodiment, the hydrogel may be a synthetic hydrogel, for example comprising or consisting of synthetic peptides, or peptide/adhesion functionalized polysaccharides.

Advantageously, the mix of collagen I and collagen IV in the hydrogel yields a high proportion of myeloid cells and a population of mesenchymal stromal cells, which are important for remodelling the bone marrow space.

Once the mesodermal aggregates are embedded into the hydrogel, vascular sprouts may form which function as primitive blood vessels.

In one embodiment, the mesoderm aggregates are allowed to sprout until an optimal size of between 800 μm and 2 mm is observed, which may be between d10-12. Once the optimal aggregate size has been reached, the organoids may be extracted from the mixed matrix hydrogel and media. The extracted organoids can be further cultured on tissue plates, such as ultra-low attachment plates (e.g. tissue culture plates or wells comprising a layer of covalently bonded hydrogel) or other tissue culture ware, and/or flow cells or chips. The extracted organoids can be further cultured under media flow conditions.

### Extraction

Sprouted bone marrow organoids may be extracted from the mixed matrix hydrogel and resuspended in basal media (Phase IV medium). The sprouted bone marrow organoids may be cultured individually, for example in multi-well ultra-low attachment dishes. The bone marrow organoids may be cultured in media, such as basal media. In one embodiment, the basal media (Phase IV medium) comprises the same media, including supplements, as the sprouting medium (Phase III medium) of step b.; or the same media, but with reduced cytokine content, such as about 50% reduction in cytokine content. The cytokine concentration may be maintained or reduced. The cytokine concentrations may be reduced to 10 ng/mL. In another embodiment, the cytokine concentrations may be reduced to 1 to 10 ng/mL.

The bone marrow organoids may be extracted from the mixed matrix hydrogel by physically/mechanically scraping them from the mixed matrix hydrogel and/or the extracted bone marrow organoids may be suspended in a media, such as basal media, and centrifuged to separate the bone marrow organoids from the mixed matrix hydrogel and form free bone marrow organoids.

The bone marrow organoids may be extracted at any suitable time, such as from day 12. The bone marrow organoids may be extracted at any suitable time, such as from day 12 to 30 or more, such as for as long as the cells of the bone marrow organoids are viable. The bone marrow organoids may be obtained at d18 or more for validation experiments. Validation may be performed by any means known to the skilled person, for example by immunofluorescence imaging of whole, ethyl cinnamate cleared bone marrow organoids, or by imaging of the bone marrow organoids embedded in optical cutting temperature (OCT) solution and frozen, or by embedding in paraffin. Validation may be performed by genetic analysis such as RNA sequencing and/or qRT PCR.

### Other Aspects

According to another aspect of the present invention, there is provided a model of a bone marrow organoid, wherein the bone marrow organoid is formed by the methods described herein.

According to another aspect of the present invention, there is provided bone marrow organoids formed by the methods described herein.

In one embodiment, the bone marrow organoids comprise haematopoietic stem/progenitor cells (CD34+), neutrophils and monocytes (CD11b+, Lin+), megakaryocytes (CD41+, CD34−, Lin−), erythroid cells (CD71+, CD235+, Lin−), endothelial cells (CD31+, CD144+), fibroblasts and bone marrow mesenchymal stromal cells (PDGFRb+, LepR+, VCAM1+). Preferably, the bone marrow organoid further comprises a vasculature network and/or sinusoidal cells. In one embodiment, the bone marrow organoid does not comprise osteoprogenitors and/or lymphoid cells. In an alternative embodiment, the bone marrow organoid comprises osteoprogenitors and/or lymphoid cells. In one embodiment, the bone marrow organoid is synthetically produced (i.e. in vitro) and does not comprise bone marrow tissue extract. In another embodiment, the bone marrow organoid is entirely comprised from cells derived from native bone marrow and assembled in the support matrix described herein.

The bone marrow organoids may further comprise cancer cells, for example from a cancer donor. The cancer cells may be engrafted cancer cells (e.g. cancer cells isolated from patients that are added to the cultures). The cancer may be a blood malignancy. The cancers cells may be from patients with myeloid or lymphoid blood malignancies, such as myeloma, acute or chronic lymphoblastic leukaemias, acute or chronic myeloid leukaemias, myelodysplastic syndrome, myeloproliferative neoplasms, lymphomas, or mast cell neoplasms. The bone marrow organoids may further comprise cells from a healthy donor, which may be engrafted. The cells from a healthy donor may be one or more of CD34+ cells, peripheral blood mononuclear cells (PBMCs), donor derived fibroblast or endothelium cells.

According to another aspect of the present invention, there is provided a model for fibrosis, wherein the model for fibrosis comprises a bone marrow organoid according to the invention that has been treated with an agent to induce collagen deposition in the bone marrow organoid.

According to another aspect of the present invention, there is provided a method of producing a model for fibrosis, wherein the method comprises treating a bone marrow organoid according to the invention with an agent that is capable of inducing collagen deposition in the bone marrow organoid.

In one embodiment, an agent is used to induce fibrosis in the bone marrow organoids. In one embodiment, an agent is used to induce extracellular matrix deposition in the bone marrow organoids, which respond to fibrosis-promoting factors by increasing smooth muscle actin and collagen expression, resulting in fibrosis. In one embodiment the agent comprises or consists of a growth factor or cytokine. The agent, such as a cytokine, may be TGFβ. In one embodiment the bone marrow organoids are treated with TGFβ to emulate fibrosis. In another embodiment, fibrosis may be induced genetically, such as by genetic modification, genetic overexpression smooth muscle actin and/or collagen, or by siRNA silencing, or by treatment with other proteins or pharmacological agents. In another embodiment, fibrosis may be induced genetically, such as by overexpression of genes, such as TGFB1 or other fibrosis-promoting genes, that induce expression of αSMA/collagen. Fibrosis may be induced by using an iPSC line from a patient suffering from fibrosis, or iPSCs may be gene edited to harbour a known fibrosis causing gene.

The amount of agent and incubation time with the agent may be an amount and time sufficient to cause collagen deposition in the bone marrow organoid, such as at least 2 ng/ml TGFβ, or such as at least 10 ng/ml, for at least 24 hours.

In another embodiment, the amount and incubation time with TGFβ may be at least 2 ng/ml, such as at least 5 ng/ml for at least 2 hours, such as at least 72 hours. In another embodiment, the amount and incubation time with TGFβ may be between about 2 ng/ml and 500 ng/ml, such as between about 5 ng/ml and 500 ng/ml, for a period of between about 2 and 96 hours or more, such as between about 24 and 96 hours or more. In another embodiment, the amount and incubation time with TGFβ may be between about 2 ng/ml and 500 ng/ml, such as between about 5 ng/ml and 500 ng/ml for a period of between about 2 and 72 hours, such as between about 24 and 72 hours. In another embodiment, the amount and incubation time with TGFβ may be between about 2 ng/ml and 50 ng/ml, such as between about 5 ng/ml and 50 ng/ml, for a period of between about 24 and 72 hours. In another embodiment, the amount and incubation time with TGFβ may be between about 2 ng/ml and 50 ng/ml, such as between about 5 ng/ml and 50 ng/ml for a period of between about 36 and 72 hours. The skilled person will appreciate that where a higher concentration is used the incubation time may be shorter, and vice versa.

According to another aspect of the present invention, there is provided the use of the model for fibrosis to identify agents capable of preventing or treating fibrosis, wherein the bone marrow organoid is treated with a potential agent before, during or after the bone marrow organoid is treated with a fibrosis-inducing agent.

In another embodiment, fibrosis induced by a fibrosis-promoting agent may be prevented by pharmacological or genetic manipulation of the organoid.

The use may comprise the determination of whether the treatment by the potential agent has any effect in inhibiting or reducing the development of fibrosis, or the reduction in fibrosis after it has developed. The use may comprise the determination of whether the treatment by the potential agent has any effect in inhibiting or reducing the deposition of collagen. The use may comprise the determination of whether the treatment by the potential agent has any effect in inhibiting or reducing smooth muscle actin and/or collagen expression, or other markers of fibrosis.

The prevention or treatment of fibrosis may comprise inhibiting, preventing or reducing fibrosis.

According to another aspect of the present invention, there is provided a method of screening for agents capable of preventing or treating fibrosis, for example using the bone marrow organoid described herein, wherein the bone marrow organoid is genetically manipulated or treated with a potential agent before, during or after the bone marrow organoid is treated to induce fibrosis; and
A) determining if the potential agent or genetic target has any effect in inhibiting or preventing the development of fibrosis in the bone marrow organoid, or the reduction in fibrosis after it has developed in the bone marrow organoid;
B) determining if the potential agent or genetic target has any effect in inhibiting or reducing smooth muscle actin and/or collagen expression or other markers of fibrosis in the bone marrow organoid; and/or
C) determining if the potential agent or genetic target has any effect in inhibiting or reducing collagen deposition in the bone marrow organoid.

The bone marrow organoids according to the present invention may be used for one or more of:
A) determining if agent(s) or potential agent(s) have any effect in reducing the survival or proliferation of cancer cells from the patient;
B) determining if agent(s) or potential agent(s) have any effect on the pathogenicity of cancer cells from the patient;
C) determining if agent(s) or potential agent(s) have any effect on reducing the pathogenic remodelling of the organoid stroma or microenvironment/niche induced by cancer cells from the patient;
D) screening for potential biomarkers of cancer in the patient from whom engrafted cells are isolated;
E) studying clonal evolution of cancer in the organoids following seeding with cells from the patient, to predict future cancer progression in the patient donor;
F) studying treatment response of cancer cells seeded in the organoids to determine the optimal treatments for the donor patients;
(G) studying the impact of a cancer on its microenvironment and/or niche; and
(H) studying the role of the microenvironment and/or niche in initiating, or promoting cancer development and/or progression.

According to another aspect of the present invention, there is provided a method for maintaining the viability of cells from a patient donor with blood cancer ex vivo, to enable mechanistic studies or screening of agents or potential agents consisting of one or more of:
A) determining if agent(s) or potential agent(s) have any effect in reducing the survival or proliferation of cancer cells from the patient;
B) determining if agent(s) or potential agent(s) have any effect on the pathogenicity of cancer cells from the patient;
C) determining if agent(s) or potential agent(s) have any effect on reducing the pathogenic remodelling of the organoid stroma induced by cancer cells from the patient;
D) screening for potential biomarkers of cancer in the patient from whom engrafted cells are isolated;
E) studying clonal evolution of cancer in the organoids following seeding with cells from the patient, to predict future cancer progression in the patient donor; and
F) studying treatment response of cancer cells seeded in the organoids to determine the optimal treatments for the donor patients.

The agent to be screened or investigated may be dosed at a physiological relevant amount. The agent to be screened or investigated may be dosed at a therapeutically relevant amount. Combinations of agents may be investigated.

The determination may be relative to an untreated bone marrow organoid (i.e. not treated with the potential agent) and/or relative to a control or reference value.

In one embodiment the agent to be investigated is a small molecule (e.g. less than 900 Da), nucleic acid, antibody therapy, cellular therapy, drug compound, metabolite or peptide. In one embodiment the agent to be investigated is a small molecule (e.g. less than 900 Da), nucleic acid or peptide. The peptide may comprise or consist of an antibody. In another embodiment the agent to be investigated is a genetic manipulation agent, such as siRNA, shRNA, CRISPR-CAS9, lentiviral or retroviral vectors, for example for over expression.

Another aspect of the invention is its ability to support engraftment and survival of cells from patients with a range of blood malignancies, including cancer cell types which are difficult to keep alive ex vivo in standard liquid culture systems. The cancer cell types may include, but not be limited to, cells from patients with myeloid or lymphoid blood malignancies such as myeloma, acute or chronic lymphoblastic leukaemias, acute or chronic myeloid leukaemias, myelodysplastic syndrome, myeloproliferative neoplasms, lymphomas, and mast cell neoplasms.

Therefore, in one embodiment, the organoids of the present invention may be used for an engraftment and/or survival assay for cells from a patient with a blood malignancy.

A method is provided for seeding of the organoids with cells from a donor, and tracking the cells to assay one or more of survival, proliferation and isolation of engrafted cells, for example for downstream functional testing. This model also presents a novel system to study cancer-associated pathogenic remodelling of the bone marrow niche, such as the fibrosis induced by a malignant clone in a proportion of patients with myeloproliferative neoplasms.

The cells maybe tracked by fluorescent markers or tags. Tracking the cells may comprise the use of a fluorescent cell tracking system.

The cell donor may be an adult or child donor.

The fibrosis in accordance with any aspect or embodiment herein may comprise or consist of myelofibrosis.

In one embodiment, the bone marrow organoids according to the invention may be used to produce blood platelets.

According to another aspect of the present invention, there is provided a method for producing platelets and/or erythroid cells (RBCs), the method comprising the incubation of bone organoids in accordance with the invention in vitro, and harvesting the platelets and/or erythroid cells (RBCs) produced from the bone organoids.

The platelets and/or erythroid cells (RBCs) may be produced naturally by the bone marrow organoids or induced, for example by the dosing of heparin, hirudin, and/or ROCKi to drive more proplatelet formation. The platelets and/or erythroid cells (RBCs) may be harvested by separating them from the cells of the bone organoids, such as by FACS. Additionally or alternatively, the platelets and/or erythroid cells (RBCs) may be harvested by BSA gradient and/or centrifugation.

According to another aspect of the present invention, there is provided a method of screening for biomarkers of fibrosis or other bone marrow disorders, the method comprising the monitoring of biomarkers released from the bone marrow organoids or cells engrafted therein, or biomarkers in tissue or cellular extracts of the bone marrow organoids.

A disease state may develop, or be induced in the bone marrow organoid, whereby changes to the biomarker profile may be determined and linked to the disease state.

The biomarkers may comprise proteins, glycoproteins, glycans, peptides, nucleic acids, or any cellular product which may indicate a diseased state of the bone marrow organoid or engrafted cells in the bone marrow organoid. The biomarkers may be cell markers, such as surface proteins.

In a further aspect, the invention provides one or more compositions comprising the recombinant growth factors and cytokines required to carry out the first aspect of the invention. The one or more composition may comprise or consist of two or more of, such as all of, recombinant BMP4, FGF2, VEGFA, Flt-3 and SCF. The one or more composition may comprise or consist of VEGFA and FGF2. The one or more composition may comprise or consist of VEGFA, FGF2, BMP4 and VEGFC. The one or more composition may comprise or consist of VEGFA, FGF2, SCF, TPO and Flt3. The one or more composition may comprise or consist of VEGFA, VEGFC, FGF2, SCF, TPO, BMP4 and Flt3. The one or more composition may comprise or consist of VEGFA, VEGFC, FGF2, SCF, TPO, EPO, BMP4, IL3, IL6 and Flt3. Any composition disclosed herein may further comprise IL7 and/or calcium.

The one or more composition may comprise recombinant BMP4 for use at a concentration of between about 10 and about 100 ng/ml, for example if diluted to a working concentration. When recombinant BMP4 is provided in the one or more composition for use at a concentration of between about 10 and about 100 ng/ml, recombinant FGF2 is provided for use at a concentration of between about 10 and about 50 ng/ml, recombinant VEGFA is provided for use at a concentration of between about 10 and about 50 ng/ml, recombinant Flt-3 is provided for use at a concentration of between about 10 and about 100 ng/ml, and/or recombinant SCF is provided for use at a concentration of between about 1 and about 100 ng/ml. Such a composition allows the skilled person to induce vascular and haematopoietic commitment of mesodermal aggregates before embedding in a hydrogel.

In another aspect, there is provided a kit comprising one or more, such as all of, recombinant BMP4, FGF2, VEGFA, Flt-3 and SCF. The kit may comprise or consist of VEGFA and FGF2. The kit may comprise or consist of VEGFA, FGF2, BMP4 and VEGFC. The kit may comprise or consist of VEGFA, FGF2, SCF, TPO and Flt3. The kit may comprise or consist of VEGFA, VEGFC, FGF2, SCF, TPO, BMP4 and Flt3. The kit may comprise or consist of VEGFA, VEGFC, FGF2, SCF, TPO, EPO, BMP4, IL3, IL6 and Flt3. Any kit disclosed herein may further comprise IL7 and/or calcium.

The kit may further comprise a set of instructions. The instructions will enable the reader to perform any method disclosed herein. The recombinant growth factors and/or cytokines may be provided in one solution in the kit, or two, three, four, five, six, seven or more separate solutions in the kit. In this way, one or more than one of the recombinant growth factors and/or cytokines may be provided in each solution, if required. The kit may also comprise a mesoderm-inducing medium, sprouting medium and/or hydrogel required to carry out any method disclosed herein.

In a further aspect, there is a method of producing hematopoietic and/or stromal cells, wherein said method comprises:
a. driving induced pluripotent stem cells (iPSCs) to form mesodermal aggregates;
b. inducing vascular and haematopoietic commitment of the mesodermal aggregates by further culturing the mesodermal aggregates in medium, supplemented with recombinant BMP4, FGF2, VEGFA, Flt-3 and SCF;
c. embedding the mesodermal aggregates in a hydrogel, and incubating the hydrogel in a sprouting medium comprising: a media suitable for stem cell maintenance supplemented with cytokines for differentiation, to form a mixture of hematopoietic and stromal cells.

The method my employ any step of any other method recited herein.

In yet a further aspect, there is provided hematopoietic and/or stromal cells obtained by any method disclosed herein.

### Definitions

References to amounts or concentrations of agents provided in the medium of incubation steps may refer to the amount or concentration provided at the start of incubation (i.e. prior to cell consumption during incubation). In another embodiment, references to amounts or concentrations of agents provided in the medium of incubation steps may refer to the total amount provided during the incubation, or the level of exposure of the agent to the cells, for example where a sustained release or drip feed system is used to continuously or periodically provide the agent during incubation.

References to “inhibition” or similar, may comprise a reduction in activity or presence of a molecule or the block of a biological pathway, such as a signaling pathway. The inhibition may be total (i.e. 100%) or at least a substantial inhibition. The inhibition may be partial inhibition. Partial inhibition may comprise significant inhibition in order to affect the desired outcome of the inhibition.

It is understood that pluripotent stem cells (PSCs) have indefinite capacity to self-renew and can differentiate into three primary germ layers of early embryo, thus differentiating into any adult cell type except extra-embryonic lineage such as placenta. Pluripotent stems cells may comprise embryonic stem cells or induced pluripotent stem cells (iPSC) made from adult somatic cells such as blood or skin cells by reprogramming technology. In one embodiment, the invention herein may not use embryonic stem cells. PSCs can be isolated or generated from non-human species. Human iPSC have the advantage harbor the patient genetic signature are useful in investigating genotype-phenotype links when differentiated into relevant cell types. This is useful for investigating disease mechanism or identifying new drug targets or investigating patient specific changes.

As used herein, the term “iPSC aggregate refers” to a 3D mass of cells, which may not grow as a spreading monolayer on a plate surface.

It is understood that “chemically defined medium” is a growth medium suitable for the in vitro cell culture of human or animal cells in which all of the chemical components are known.

It is understood that the terms “cell induction” or “cell differentiation” is the promotion of an iPSC to differentiate into a particular cell type, such that it is no longer pluripotent. The induction/differentiation may comprise the stimulation, upregulation or downregulation of specific biological pathways, which may be provided by growth conditions; agents; delivery of genes by an expression system such as plasmids; activation of genes using genome engineering approaches, reduction or knock-out of genes by an expression system such as RNAi or genome engineering approaches; or media components. It is understood therefore that the term “mesodermal induction” refers to the promotion of a pluripotent stem cell to differentiate into mesodermal tissue.

As used herein, the term “bone marrow organoid” may be taken to describe a 3-dimensional, multi-lineage cellular structure. The structure is composed of human, iPSC-derived cell types with high homology to those found in bone marrow, including stromal cells (comprised of but not limited to mesenchymal stromal cells, fibroblasts), endothelial cells, and hematopoietic cells (including but not limited to hematopoietic stem and progenitor cell types as well as myeloid cellular subtypes). The cells making up the structure are organized in highly reproducible fashion including but not limited to lumen-forming vessels with perivascular stroma and myeloid cells. The organoids can be generated in vitro, reproducibly at scale, for in vitro or in vivo experimentation e.g. to test candidate or tool compounds, cellular therapies, with or without engraftment of cells isolated from blood or bone marrow of human donors. A bone marrow organoid as defined herein may contain engrafted cells from a cancer patient or healthy patient.

Unlike naturally occurring bone marrow, bone marrow organoids are grown ex vivo, in miniature and in multiples, typically forming multiple structures that are a millimeters in diameter, such as the size of between 800 μm and 5 mm, or between 800 μm and 2 mm. The bone marrow organoids may be less than 5 mm, or less than 3 mm in size (measured as the largest diameter). In contrast, human bone marrow exists in large bony structures. A key distinguishing feature of bone marrow organoids is that they are grown reproducibly with multiple replicates, enabling experimental studies. In normal human physiology, the bone marrow is populated by cells from the fetal liver during gestation. In the present system, we differentiate mesodermal and bipotent hematopoietic-endothelial progenitors from iPSCs, which form hematopoietic, vascular as well as stromal lineages in the organoid within the culture system. Using iPSCs and differentiating the cell lineages in a dish enables multiple, reproducible replicates of bone marrow organoids for experimental manipulation which incorporate haematopoietic, stromal and vascular elements. This is not possible using native human tissue. The iPSC-derived bone marrow organoids can contain cell types that are transcriptionally representative of fetal liver, fetal bone marrow and adult bone marrow cells, in contrast to human tissues.

The skilled person will recognise that the term “organoid” may alternatively be termed “spheroid” or “microtissue”.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

### FIGURE REFERENCES

FIG. 1: Development of a protocol for a matrix-dependent differentiation of an iPSC derived bone marrow organoid. A step wise differentiation protocol for the generation of a bone marrow organoid was developed in which undifferentiated iPSCs are first detached and cultured on ultra-low attachment plate to form aggregates. (A) Undifferentiated iPSC organoids are subsequently subject to a Phase I medium for mesoderm induction at 5% O2. This medium is comprised of APEL2 supplemented with BMP4, FGF2, VEGFA Mesodermal aggregates are grown until they reach an average size of 200-250 um (typically day 3), before being collected by gravitation and resuspended in Phase II medium for vascular and haematopoeitic commitment. Once aggregates are cultured for 48 hours, or have achieved an average size of 350-400 um, they are embedded in mixed matrix hydrogels (Matrigel supplemented with Collagen I, Collagen IV, or a combination of both). Cells grown as rounded aggregates on ultra low attachment plates as indicated in panel A. Once within these matrices (B) cells are cultured in Phase III medium until sprouts achieve a maximal size (typically between 800 um and 1.5 mm, typically on day 10-12) before they are extracted from their hydrogels and resuspended as individual organoids in each well of a multiwell plate (e.g. a 96-well plate or 384-well plate). Sprouts demonstrated in panel B as cell out growth from the central spherical aggregate within a hydrogel.

FIG. 2: Validation of a matrix dependent differentiation of an iPSC derived bone marrow organoid. (A) A stepwise differentiation protocol comparing the generation of bone marrow lineages in a Collagen I, Collagen IV, and Collagen I+IV (60%) and matrigel (40%) hydrogel. This protocol generates sprouting cultures which are isolated and allowed to mature to form large 3D vascularised compartments. (B,C) Flow cytometry based validation of bone marrow organoid populations at d16. Mature bone marrow organoids were dissociated for flow cytometry, and key stromal and haematopoeitic populations were identified across the different hydrogel mixes used. Distinct endothelial (CD31+CD144+) populations were found across materials, notably however, a bone marrow mesenchymal stromal cell (MSC) population was only present in gels containing Collagen IV (LepR+VCAM1+CD140b+). A distinct CD34+ population was also observed in all three matrices, however significant increases in megakaryocytic (CD41+CD34−Lin−) and erythroid (CD71+CD235+Lin−) lineages were observed in the presence of collagen IV. (C) The most notable differences in organoid composition were in the increased presence of myeloid cells (such as monocytes/neutrophil), erythroid and megakaryocyte cells in the presence of Collagen IV, as well as a population of LepR+ MSCs in Collagen IV-containing cultures.

FIG. 3: Distinct hydrogel specific sprouting behaviours in bone marrow organoids. A stepwise differentiation protocol comparing the generation of bone marrow lineages in a Collagen I, Collagen IV, and Collagen I+IV (60%) and matrigel (40%) hydrogel. This protocol generates sprouting cultures which are isolated and allowed to mature to form large 3D vascularised organoids. (A,B) Immunofluorescence imaging of sprouts at d12 stained for key stromal (CD140b), endothelial (CD144), and haematopoeitic cells (CD34 and CD41) shows the formation of branching vasculature and associated CD34+ haematopoeitic cells and mature bone marrow resident megakaryocytes (CD41+ cells). Positive staining in these images indicates the presence of the labelled lineages within the images. In panel B, CD41+ cells are either rounded megakaryocytes, or cells forming long proplatelet extensions as found in vivo.

FIG. 4: Addition of VEGFC induces specialisation of organoid endothelium to resemble bone marrow sinusoidal endothelium. The protocol thus far generated an organoid producing the key lineages of the central human bone marrow. However, bone marrow sinusoidal endothelium, is a highly specialised for regulating haematopoeitic cell behaviours. We determined that the addition of VEGFC to VEGFA in the growth factor cocktail improved the expression of adhesion molecules and hematopoietic regulatory factors to be more similar to native human bone marrow, compared to endothelial cells generated in cultures with VEGFA or VEGFC alone, including in VEGFR3, VCAM1, VLA4, FGF4, CXCR4.

FIG. 5: VEGFC induces development of CD34+ sinusoidal vascular network in organoids. (A) Addition of VEGFC to culture medium induces vessels in the organoid that maintain the expression of CD34, similar to adult bone marrow sinusoidal endothelium. Immunofluorescence images show significant staining of the vessels in the VEGFA+C treated sample when compared to VEGFA. (B) Close association of CD41+ megakaryocytes with vessels in the organoids. Inset shows rounded CD41+ megakaryocytes. (C) CD41+ megakaryocytes are shown forming pro-platelet extensions in sections of BM organoids. Bottom right panel demonstrates rounded cells emerging from megakaryocytes associating with vessel.

FIG. 6: Volumetric imaging and rendering of bone marrow organoids: Mature bone marrow organoids were fixed and stained for stromal markers (UEA1 for branching endothelium, PDGFRβ for fibroblast/MSC) and pro-platelet forming megakaryocytes. Samples were cleared and imaged using a Zeiss LSM 880 confocal microscopy, before processing and rendering in Imaris. The figures show a complex vascularised compartment rich in supporting fibroblast/MSC and proplatelet forming megakaryocytes, effectively mimicking key elements of the bone marrow microenvironment.

FIG. 7: Treatment of organoids with TGFβ induced fibrosis of the organoids, modelling bone marrow fibrosis seen in patients. (A) Following 72-hours of stimulation with TGFβ, samples were fixed and stained for α-smooth muscle actin (α-SMA) and collagen I (COL1A1) show an increase in the expression of these proteins compared to an untreated control. Increase of signal in these images is indicative of treatment inducing classic markers of fibrosis. (B) These differences are quantifiable by qRT PCR which shows a dose dependent increase of canonical fibrosis markers, indicating the efficacy of this model for modelling bone marrow fibrosis. Finally, we confirmed the utility of this model to screen for potential inhibitors of bone marrow fibrosis. Addition of two compounds—SB431542, an inhibitor of the TGFβ superfamily type I activin receptor, and the bromodomain inhibitor JQ1—significantly reduced TGFβ-induced collagen deposition and αSMA expression. (N=4 and 3 respectively, significance testing with Kruskal-Wallis with multiple comparisons).

FIG. 8: Seeding and engraftment of organoids by healthy and malignant cells from patients: (A) Cells from adult human donors were labelled with CellVue prior to seeing, to enable tracking and distinction from iPSC-derived organoid cells. Confocal imaging of engrafted organoids show penetration of donor cells (in grey) within the organoid volume, and homing of the cells to branching vessels (magenta—UEA1 labelled vasculature). Within the right hand side inset, labelled cells are derived from patients and populate the volume of the organoid as demonstrated by this maximum intensity projection of CellVue+ cells. (B) Whole organoid labelling and imaging shows effective engraftment of cells from one healthy donor and two patients with myelofibrosis. Signal in the right hand column of this image shows labelled patient derived cells within the volume of the organoid. (C) Organoids engrafted with cells from myelofibrosis patients show a marked increase in collagen I and α smooth muscle actin (αSMA), hallmarks of fibrosis. Schematic created with Biorender.com.

FIG. 9: H&E staining reveals lumen-forming vasculature within organoids with blood cells extravasating into vessel lumens. Hematoxolin and eosin stain of a section of a bone marrow organoid shows lumen formation and the accumulation of cells, including red blood cells and myeloid progenitors within the vessel lumen. Within the left hand cropped zoom figures indicate luminal spaces occupied by blood cells.

FIG. 10: Cells isolated from healthy donors (A, top) and patients with a variety of types of blood cancer (A, middle—infant acute lymphoblastic anaemia [iALL]; A, bottom—chronic myeloid leukaemia [CML]; B—multiple myeloma [MM]) were labelled with CellVue and seeded into wells containing organoids. The cells homed to and engrafted the organoids, visible throughout the organoid body. Cells within the CellVue labelled panel are all patient derived cells from the described cancers. (C): Cells in media alone died rapidly (<10% viability at day 8) whereas cells were >80% viable at day 8 when supported by organoids.

FIG. 11: Organoids engrafted with CD34+ cells from patients with myelofibrosis (MF) are extensively remodeled, reconstituting hallmarks of the disease. (A,B) Here patient engrafted and unengrafted controls are embedded into paraffin blocks and sectioned with routine, clinical histological stains used to assess the bone marrow of patients with myelofibrosis. Compared to unengrafted controls, bone marrow organoids engrafted with cells from patients with myelofibrosis demonstrate no notable change in size (measured as diameter of H&E stained sections), but a marked increase in the percentage area of reticulin staining, and a significant decrease in the percentage area of vascular staining. Reticulin staining is used clinically to establish fibrosis in patients, and the induction of reticulin fibrosis in patient engrafted samples demonstrates that patient cells are not only able to successfully transplant BM organoids, but that they are able to induce hallmarks of fibrosis allowing for effective and unprecedented modelling of this exemplar blood cancer in vitro. The loss of vascular staining indicates extensive remodeling of the niche across lineages.

FIG. 12: Organoids engrafted with CD34⁺ cells from patients with myelofibrosis can be used to screen potential therapies in a patient specific approach. Bone marrow organoids were seeded with cells from patients with myelofibrosis for 14 days in total, with pharmacological interventions staged at day 7 post engraftment. Compared to unengrafted controls, DMSO treated MF-patient organoids demonstrated increases in hallmarks of fibrosis (COL1A1, αSMA) as measured by immunofluorescence imaging of paraffin embedded sections (A). Samples were also treated with the inhibitor SB431542, the BET bromodomain inhibitor JQ1, and the current clinical treatment for myelofibrosis ruxolitinib (at 0.1 and 1 uM respectively). While significant reductions in COL1A1 were observed in JQ1 and ruxolitinib treated samples, only JQ1 significantly reduced both COL1A1 and αSMA levels to pre-fibrotic levels. (B) Representative immunofluorescence imaging of paraffin embedded sections. This data shows that patient engrafted bone marrow organoids can be used to test existing and potential therapeutic agents in personalized/precision medicine and patient specific approach.

FIG. 13: Flow cytometry confirms functionality of patient engrafted cells after 2-weeks of culture in BM organoid. (A) A flow cytometry panel investigating the fate of patient CD34+ cells engrafted into bone marrow organoids. After 2-weeks of culture, organoids containing patient cells were dissociated and subject to flow cytometry. Cells were gated on CellVue staining to discriminate patient cells from background organoid cells. Costaining with key markers demonstrates that CellVue+ cells differentiate in myelomonocytic, erythroid, and megakaryocytic cells at two weeks post-culture. (B) Labelling patient cells with the proliferation dye CellTrace and CD34 demonstrated that within the cultures a population of quiescent CD34+ patient cells were retained. This data demonstrates the functionality of patient cells within the BM organoid, including the expansion and proliferation of CD34+ hematopoietic stem and progenitor cells.

FIG. 14: Bone marrow organoids support cells from xenografts from a murine infant Acute Lymphoblastic Leukemia model. Cells derived from a murine xenograft model were obtained and labelled with the proliferation dye CellTrace to assess survival and proliferation in both BM organoid and the current gold standard, 3D primary bone marrow mesenchymal (3D BM-MSC) co-cultures. (A,B) We demonstrate significantly improved viability and proliferation of donor cells in bone marrow organoid cultures when compared to 3D BM-MSC co-cultures over a 12 day culture period (with samples assessed at days 2, 5, 7 and 12)@ We also demonstrate a significantly increased retention of phenotype at day 12 as measured by the percentage of CellTrace+ cells retaining CD19+ signal.

FIG. 15: Bone marrow organoids support cells patients with acute lymphoblastic leukaemia. Cells from patients with acute lymphoblastic leukaemia were obtained and labelled with the proliferation dye CellTrace to assess survival and proliferation in BM organoids, liquid cultures and the current gold standard, 3D primary bone marrow mesenchymal (3D BM-MSC) co-cultures. (A,B) We demonstrate significantly improved viability and proliferation of donor cells in bone marrow organoid cultures when compared to 3D BM-MSC co-cultures and liquid cultures over a 12 day culture period (with samples assessed at days 2, 5, 7 and 12) (C) We also demonstrate a significantly increased retention of phenotype at day 12 as measured by the percentage of CellTrace+ cells retaining CD19+ signal.

FIG. 16: Bone marrow organoids support cells patients with acute lymphoblastic leukaemia. Cells from patients with multiple myeloma were obtained and labelled with the proliferation dye CellTrace to assess survival and proliferation in BM organoids, liquid cultures and the current gold standard, 3D primary bone marrow mesenchymal (3D BM-MSC) co-cultures. (A,B) We demonstrate significantly improved viability and proliferation of donor cells in bone marrow organoid cultures when compared to 3D BM-MSC co-cultures and liquid cultures over a 12 day culture period (with samples assessed at days 2, 5, 7 and 12) (C) We observe no significant change in the percentage of patient cells measured as CD38+ CellTrace+.

FIG. 17: Bone marrow organoids secrete hematopoietic and stromal growth factors. A washout experiment was performed where all exogenous growth factors were removed from bone marrow organoid culture medium for 12 days. Media was replaced at a 50:50 ratio every 72 hours, and at day 12 supernatant was collected and assayed using a Luminex kit to detect the secretion of key growth factors at the protein level. In the absence of exogenous supplementation, key hematopoietic growth factors were detected at significant levels, including but not limited to KITLG, FLTL, Interleukin, CCL and CXCL family member chemokines.

FIG. 18: Single Cell RNA Sequencing confirms distinct hematopoietic and stromal populations found in human bone marrow. Single cell RNA sequencing was performed on bone marrow organoids to identify and characterize cell types produced within these cultures. (A) 9,516 cells were sequenced and analyzed to identify a hematopoeitic and stromal compartment. Hematopoietic cells included hematopoietic stem and progenitor cells (HSPCs), platelet producing megakaryocytes (MKs), erythroid cells at different stages of maturation, monocytic cells, and myeloid progenitors (distinct Eo/Baso/Mast progenitors and Monocyte/Neutrophil progenitors. (B) A comparison to gene sets derived from published data from primary human blood cells shows a high degree of homology through gene set enrichment analysis (GSEA) which confirms close homology to primary human cells. (C) Ligand-receptor interaction mapping of single cell data reveals extensive autocrine and paracrine interactions across the microenvironment recapitulated by the bone marrow organoid system.

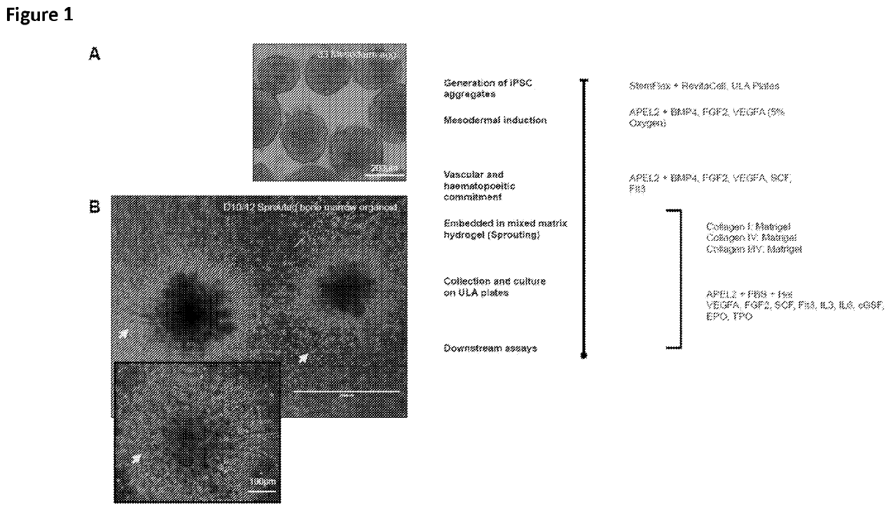

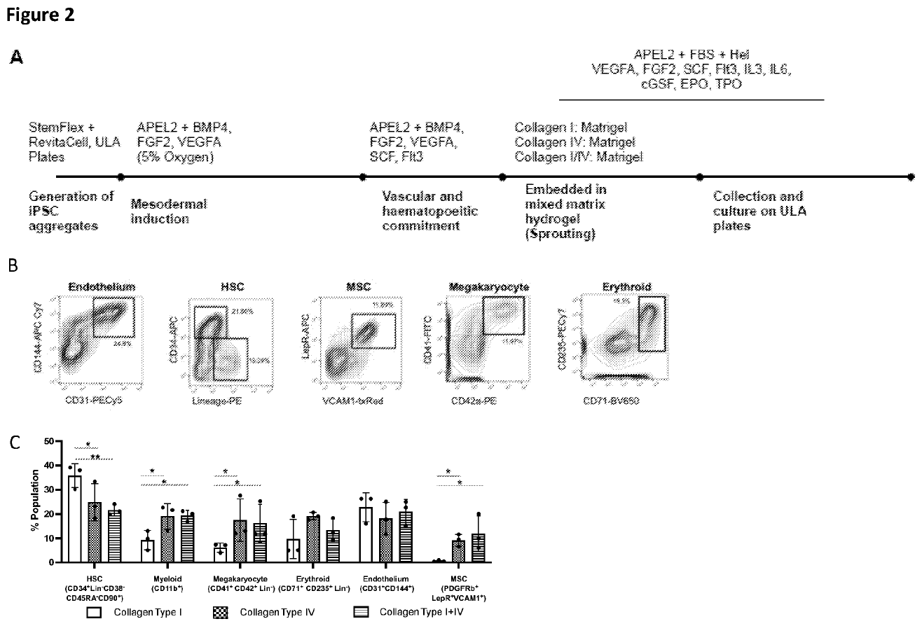

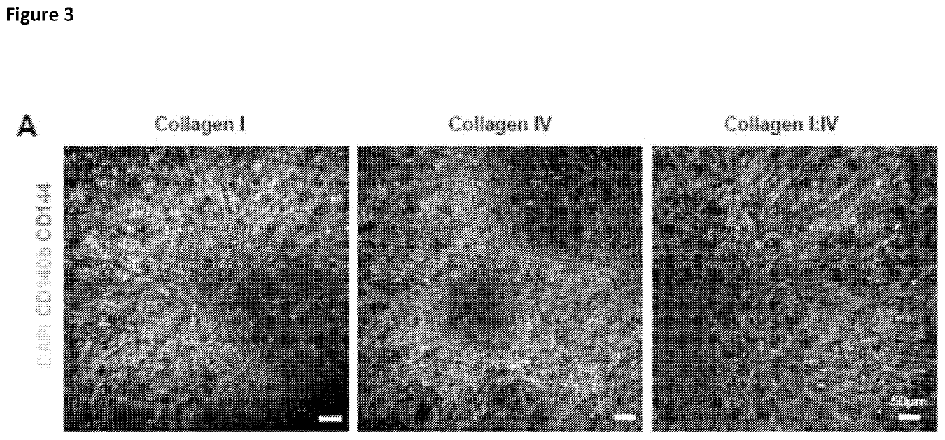

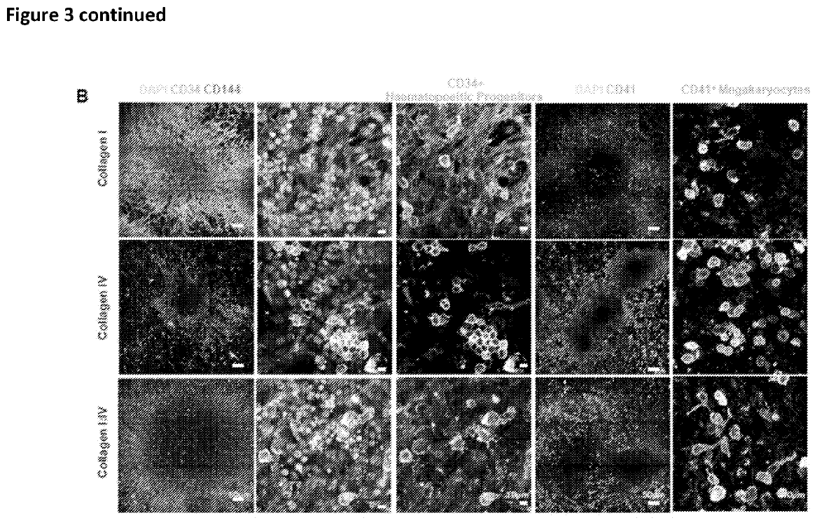

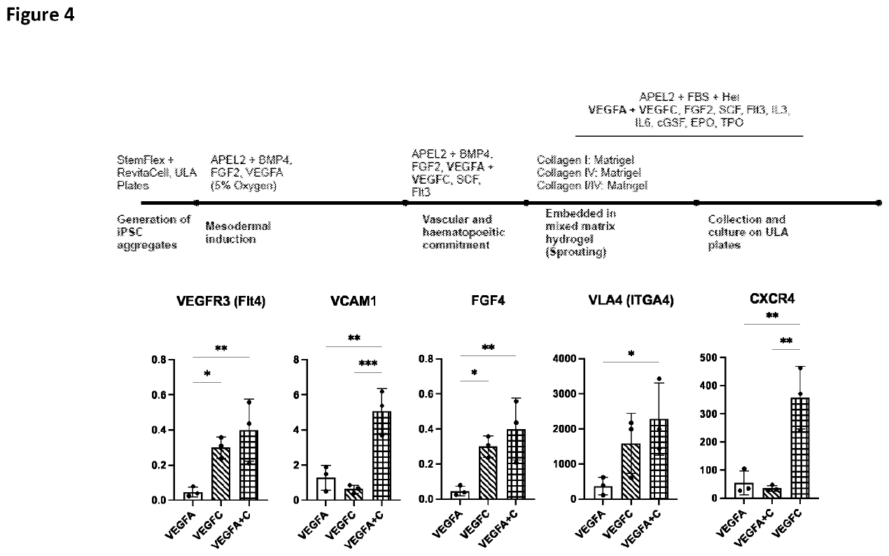

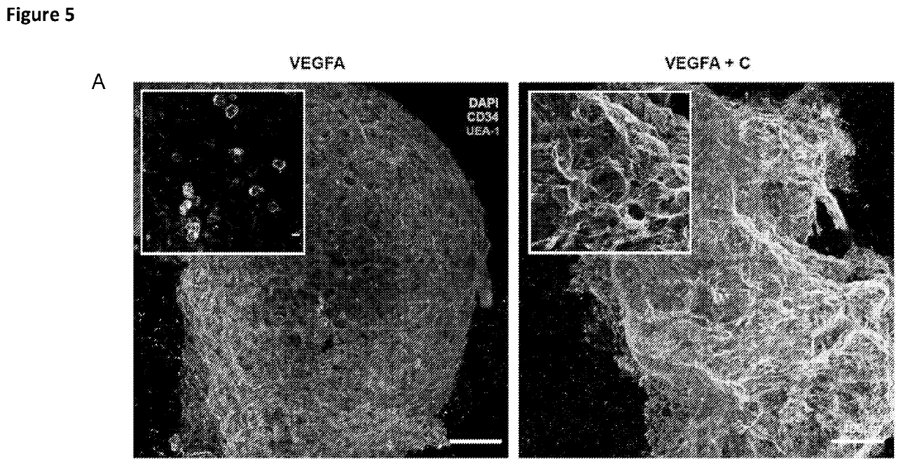

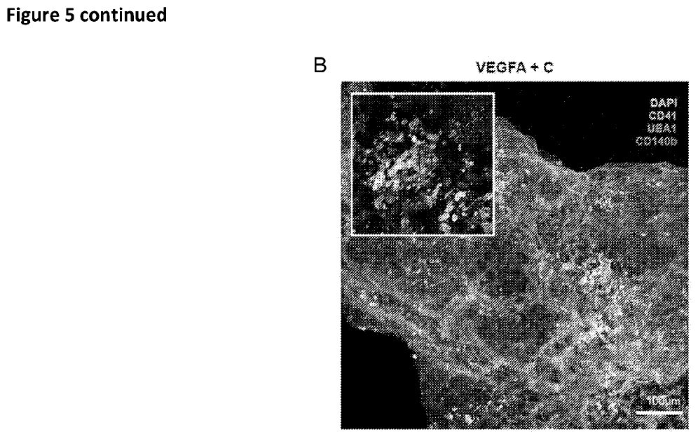

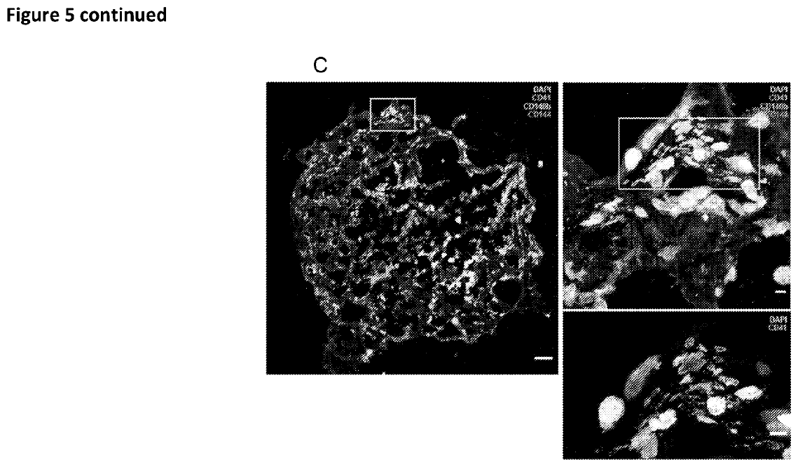

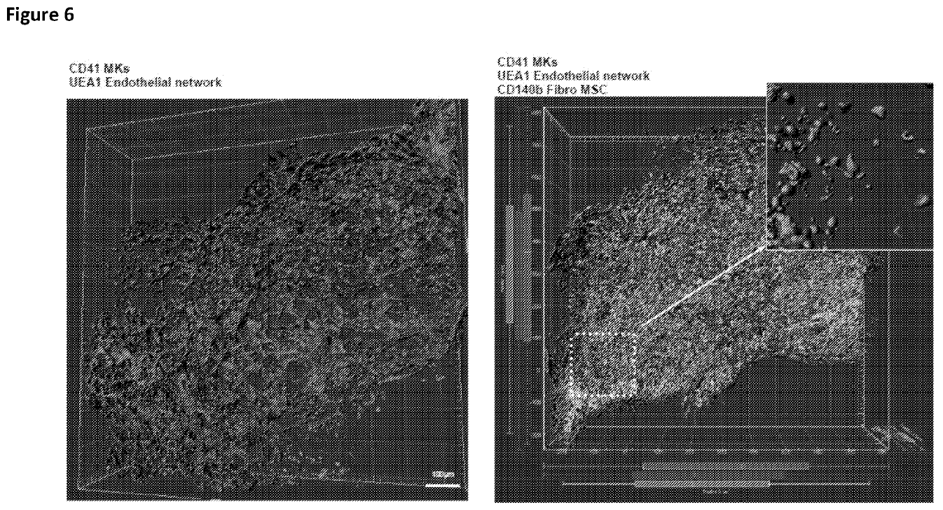

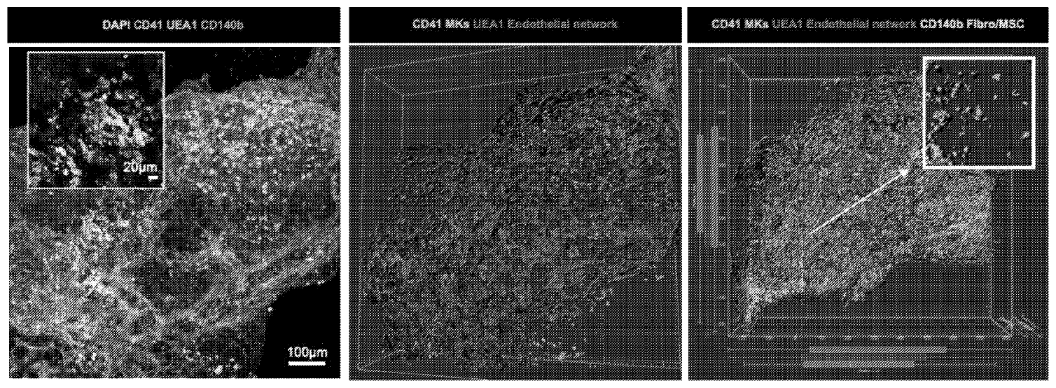

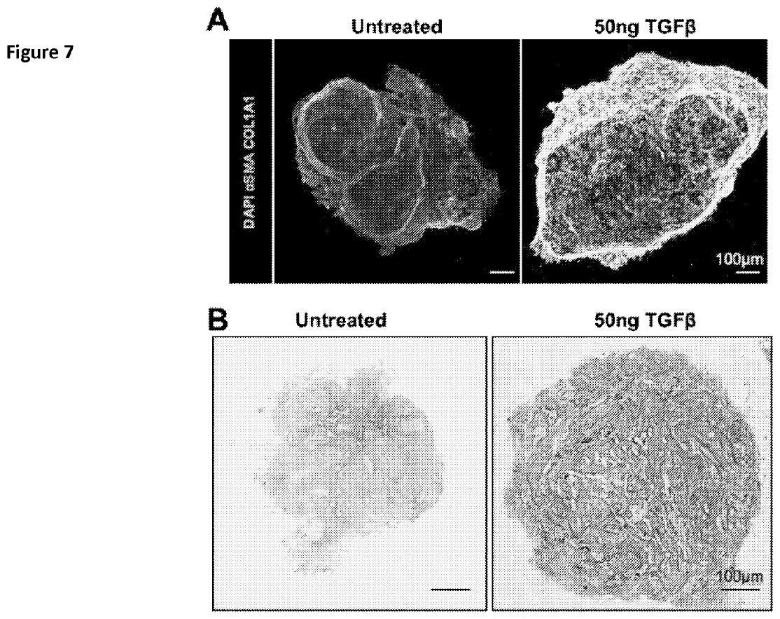

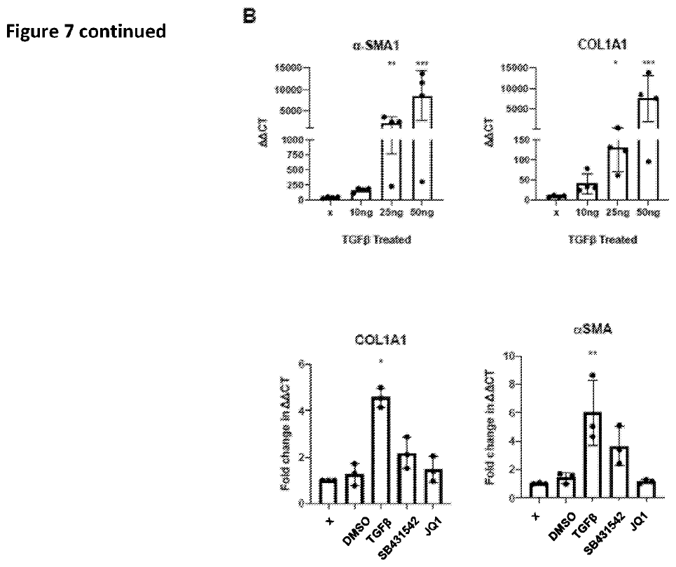

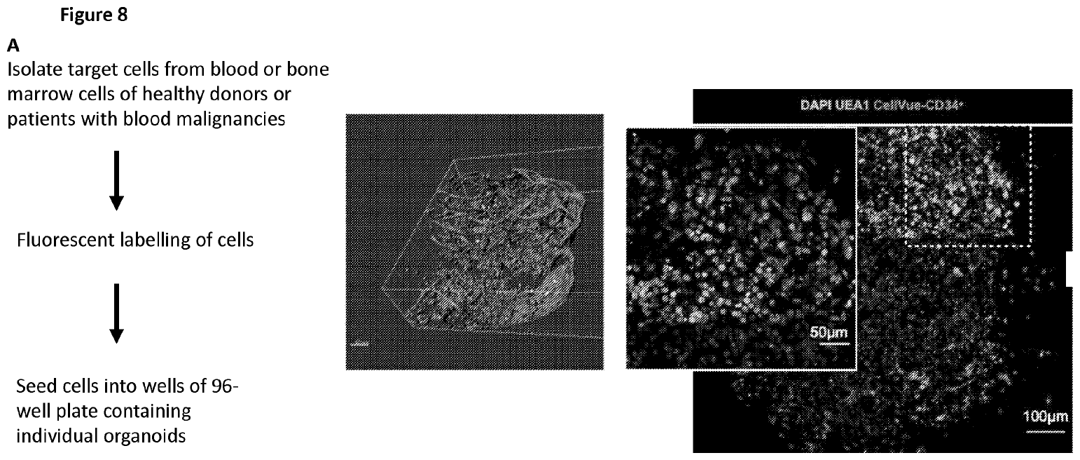

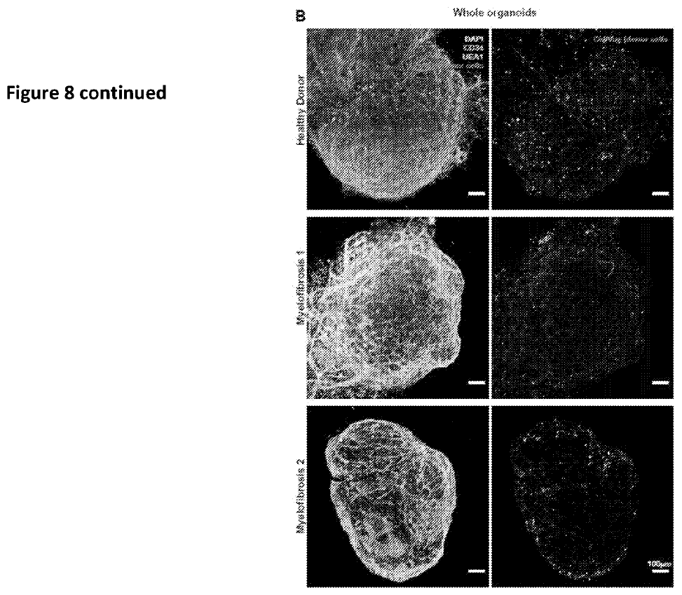

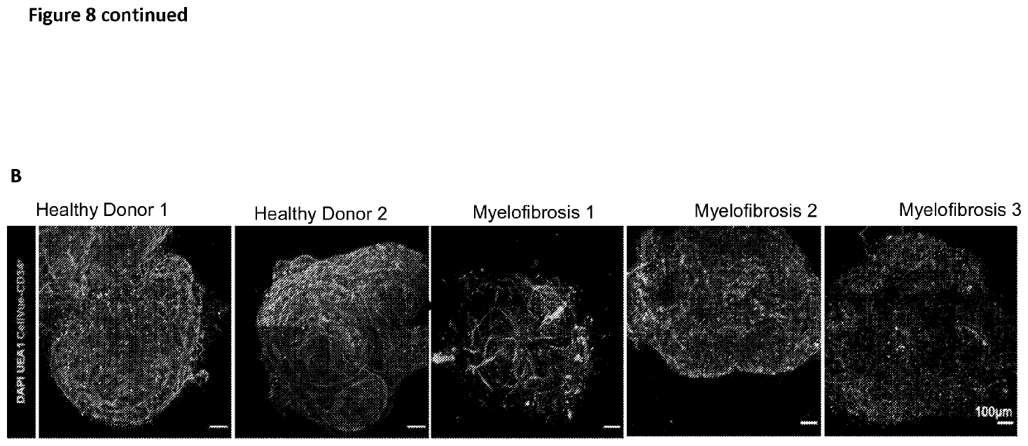

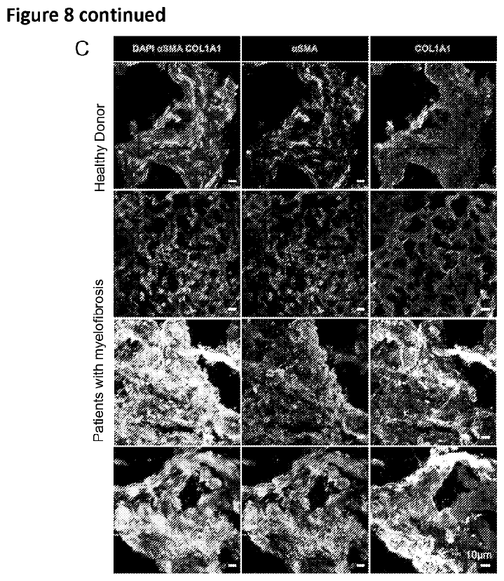

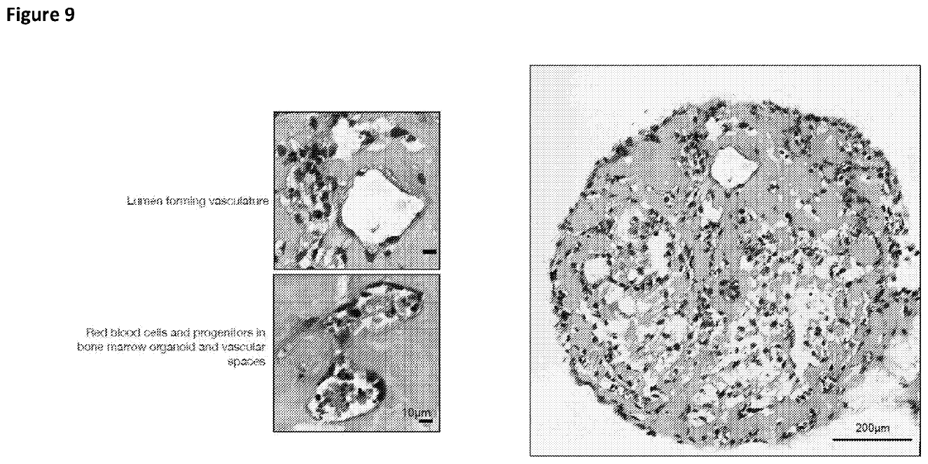

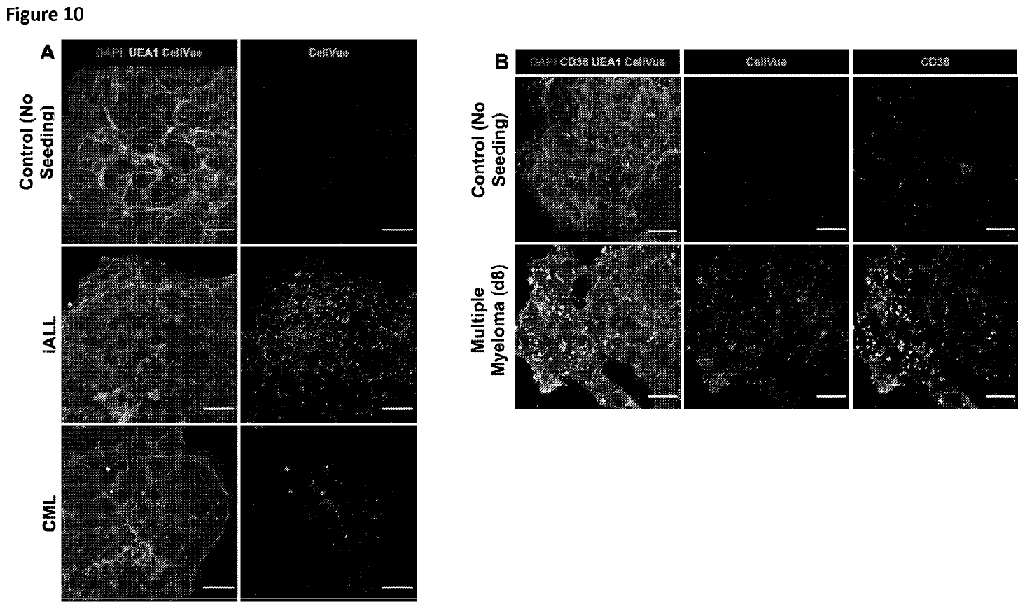

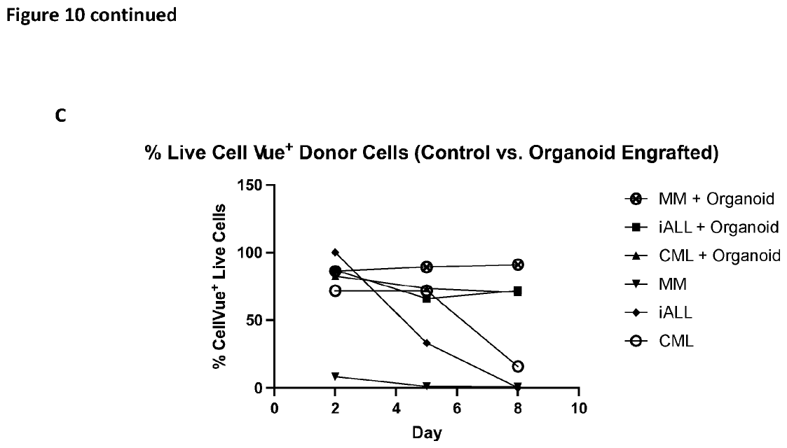

### EXAMPLES, MATERIALS AND METHODS

### iPSC Culture and Maintenance

A human induced pluripotent stem cell line (iPSCs) purchased from Gibco (Thermo) was cultured on GelTrex (Thermo) coated 6-well tissue culture plates (Corning). Cells were passaged at approximately 70% confluence using EDTA detachment. Briefly, wells were washed once with PBS and once with EDTA before a 3-minute incubation at 37° C. and 5% CO₂. EDTA was then aspirated, and cells removed through gentle mechanical dissociation by pipetting with StemFlex (Thermo) basal media. Cells were then diluted and maintained in StemFlex medium.

### Differentiation Protocol

A step wise differentiation protocol was applied to generate vascularised bone marrow organoids. Firstly, baseline iPSCs grown to 70-80% confluence before detachment using the EDTA method described above. Detached iPSCs were cultured overnight in StemFlex supplemented with

RevitaCell (Thermo) on 6 well ultra-low attachment plates. The resulting iPSC aggregates were then collected either by gravitation or low speed centrifugation on the following day (d0), and resuspended in Phase I medium. Phase I medium was comprised of APELII (Stem Cell Technologies) supplemented with 50 ng/ml of Bone Morphogenic Protein-4 (BMP4) (Thermo), Fibroblast Growth Factor-2 (FGF2) (Stem Cell Technologies) and Vascular Endothelial Growth Factor A (VEGFA-165) (Stem Cell Technologies). Cells were then maintained at 5% O₂, 5% CO₂, and 37° C. for 72 hours until mesodermal aggregates reached an average size of approximately 200-250 um (typically d3).

After 72 hours, cells were collected via gravitation before resuspension in Phase II medium, which was comprised of APELII with 50 ng each BMP4, FGF2, and VEGFA, supplemented with 25 ng Fms Related Receptor Tyrosine Kinase-3 (Flt-3) and stem cell factor (SCF) (25 ng/mL). Cells were cultured for a further 48 hours, or until they achieve an average size of 350-400 um, under these conditions (typically d5).

At d5 cells were collected via centrifugation and prepared for embedding in mixed matrix hydrogels. Initially, different compositions of hydrogels were tested to determine the best conditions for the generation of myeloid and bone marrow specific lineages. Each matrix was comprised of 40% reduced growth factor Matrigel (Corning) and 60% either Collagen Type I or Collagen Type IV (Cell Systems), or a mixed Collagen I Collagen IV gel. All gels were prepared with Collagen at a concentration of 1 mg/mL. Gel preparation began on d4 when Matrigel aliquots were thawed overnight at 4° C. Gel mixtures were prepared on ice, with Collagen mixes neutralised with 1N NaOH prior to distribution in 12 well cell culture plates. Each hydrogel was allowed a minimum of 90 minutes to polymerise. An initial cell free layer was prepared before cells were collected by gravitation and resuspended in the remaining gel volume.

Once fully polymerised, 3D cultures were supplemented in Phase III sprouting medium which was comprised of APELII medium supplemented with 5% Foetal Bovine Serum (FBS), 5 U/mL Heparin Sulfate, 50 ng VEGFA, 10 ng each Interleukin 3 (IL3) and Interleukin 6 (IL6), as well as 25 ng each of SCF, Flt3, Thrombopoietin (TPO), Erythropoietin (EPO), Granulocyte Colony Stimulating Factor (G-CSF) (Stem Cell Technologies), FGF2, and BMP4. Cells were maintained in this media formulation until d12, with media changes every 72 hours. From d5 onwards, media changes are performed as 60:40 splits fresh: conditioned medium. For experiments developing sinusoid specific endothelial vasculature Vascular Endothelial Growth Factor C (VEGFC, Stem Cell Technologies) was supplemented into d3 media.

Cultures are allowed to sprout until an optimal size of between 800 um and 1.5 mm is observed, typically between d10-12. At this stage sprouted bone marrow organoids were extracted from hydrogels, resuspended in Phase IV medium, and cultured individually in 96-well ultra-low attachment dishes. Organoids were harvested first by scraping with a sterile cell scraper, before pipetting in an excess of media into a 15 mL Falcon (Corning). Samples were then spun down at 500 G for 5 minutes to separate organoids from both the media and collagen. The free organoids are then resuspended in the desired volume of media and collected for individual culture in 96-well ultra-low attachment plates. Phase IV medium was formulated similarly to Phase III medium, however with all cytokine concentrations reduced to 10 ng/mL.

Organoids were obtained at d18 for validation experiments. Validation was either performed by immufluorescence imaging of whole, ethyl cinnamate cleared organoids, or imaging of organoids embedded in optical cutting temperature (OCT) solution and frozen.

### TGFβ Treatments

To model myelofibrosis (MF), we treated bone marrow organoids with Transforming Growth Factor β (TGFβ, Peprotech) at various concentrations for 72 hours. Treated organoids were maintained in this media before collection via pasteurette and fixation for immunofluorescence (whole organoids or embedded sections), or centrifugation for RNA extraction and subsequent quantitative Real Time Polymerase Chain Reaction (qRT PCR assessment of changes in gene expression). RNA extraction was performed using the Qiagen RNAEasy micro kit to the manufacturer's instructions.

### Flow Cytometry

Flow cytometry was performed using CyAn ADP High-Performance Flow Cytometer. Samples were dissociated using Collagenase Type B (Sigma) at 20 mg/mL in sterile HEPES. Samples were collected by gravitation in a 15 mL falcon tube before washing first in PBS, then in HEPES. Once washed, samples were incubated in the prepared Collagenase solution at 37° C. for 10 minutes before complete dissociation via tituration. Single cell suspensions were washed spun at 500 G and blocked in 0.5% BSA for 15 minutes before labelling with flow cytometry antibodies.

### qRT-PCR

qRT-PCR was performed on an Applied Biosystems 7500 Fast Real Time PCR System. Primers were obtained from Integrated DNA Technologies (IDT) as pre-validated PrimeTime qPCR primers. Reactions were prepared from lug of isolated RNA converted to cDNA with the High Capacity cDNA Reverse Transcription Kit (Applied Biosystems) under manufacturer recommended thermal cycling conditions. Synthesised cDNA was diluted 5-fold and stored at −20° C. until required.

A master mix was prepared for each qPCR reaction which was made up of 10 μl TaqMan® Universal Primer MasterMix (thermos), 1 μl of forward and reverse primers, and 2 μl of cDNA at a final concentration of 5 ng/mL. Calculations for fold changes in expression were performed using the ΔΔCT method, with human GAPDH used as a housekeeping gene, and undifferentiated iPSC used as controls for expression levels.

### Immunofluorescence and Microscopy

Immunofluorescence was performed using a Zeiss LSM880 confocal microscope (25×0.8 LD LCI plan apo 0.8 dual immersion objective). Samples were prepared first by fixation in 10% formalin, before 3×5 min successive washes with PBS. Samples were then blocked overnight in a detergent blocking solution comprised of 2% goat serum (Thermo), 1% Bovine Serum Albumin (BSA) in 50 mL of PBS. 250 μl of Triton X100 and Tween-20 (Sigma) were added, as well as 500 μl sodium deoxycholate (Sigma) to permeabilise samples and encourage antibody preparation. Primary antibody incubations were performed overnight at 4° C., before 4×5-minute successive washes in PBS. Samples were then incubated overnight once more at 4° C. in secondary antibody mixes: DAPI for nuclear labelling, Alexa-488, Alxea-568, and Alexa-647 (Thermo) depending on the species mix of antibodies present.

Once labelled samples were embedded in a small volume of 0.5% agarose in Ibidi 8-well slides (Ibidi). Once cooled, this gel was subject to a progressive dehydration by a series of Ethanol washes adjusted to pH 9. Finally, the sample was dehydrated completely in absolute Ethanol before clearance with Ethyl Cinnamate (SLS). At this stage samples were ready for confocal imaging as described above.

### Seeding of Organoids With Primary Cells From Adults

Cells were isolated from cryopreserved peripheral blood or bone marrow mononuclear cells and stained with CellVue Claret Far Red Fluorescent Cell Linker Mini Kit for General Membrane Labeling (Sigma Aldrich, Cat #MINCLARET-1KT) following the instructions provided by the manufacturer. Organoids were seeded with 5000 cells per wells and cultured for 8-14 days in StemPro. On collection day, organoids were fixed for imaging or digested for assessment by flow cytometry or RNA extraction and qRT PCR.

### Media

mTeSR1 medium may comprise basal medium supplemented with recombinant human basic fibroblast growth factor (rh bFGF) and recombinant human transforming growth factor β (rh TGFβ). APEL2 medium is used for cellular differentiation protocols. APEL2 low insulin medium is used for long-term cell culture. StemPro-34 medium is used for patient cell culture and engraftment protocols. StemFlex medium (with and without Revitacell) is used for iPSC growth and/or expansion).

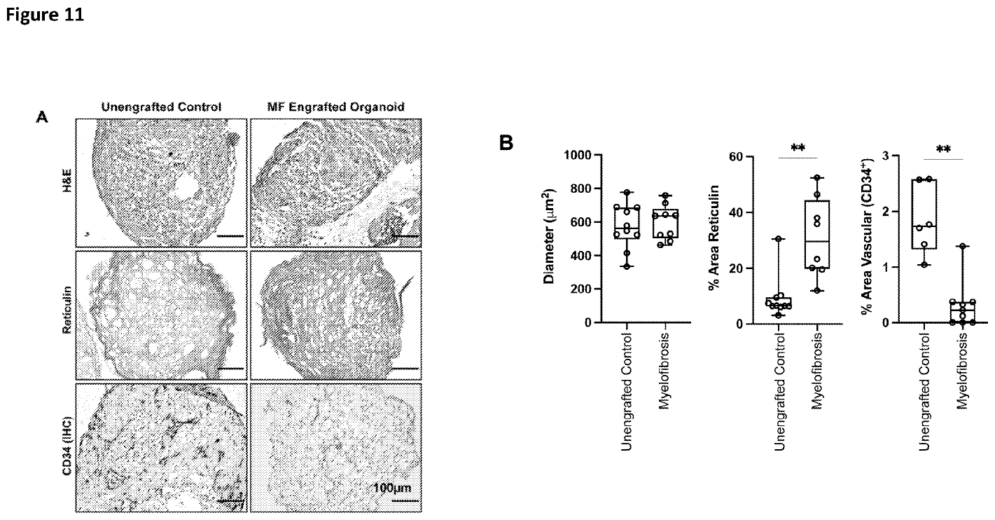

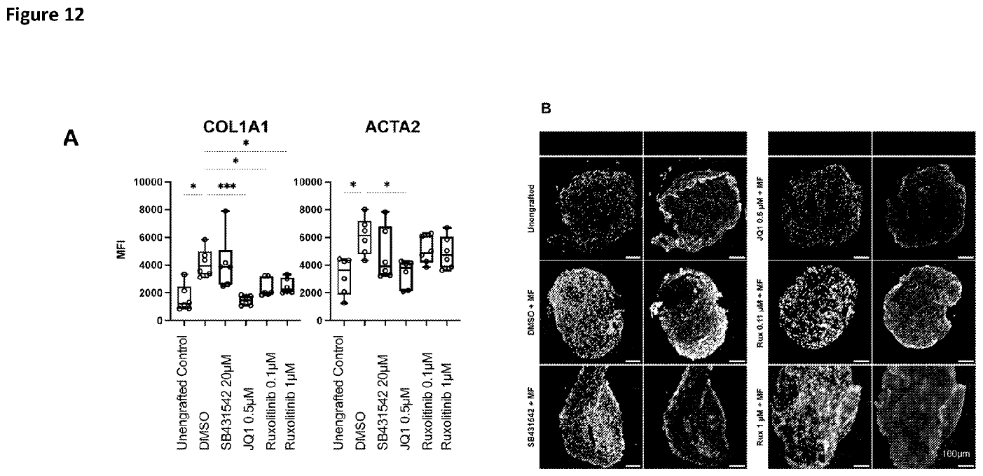

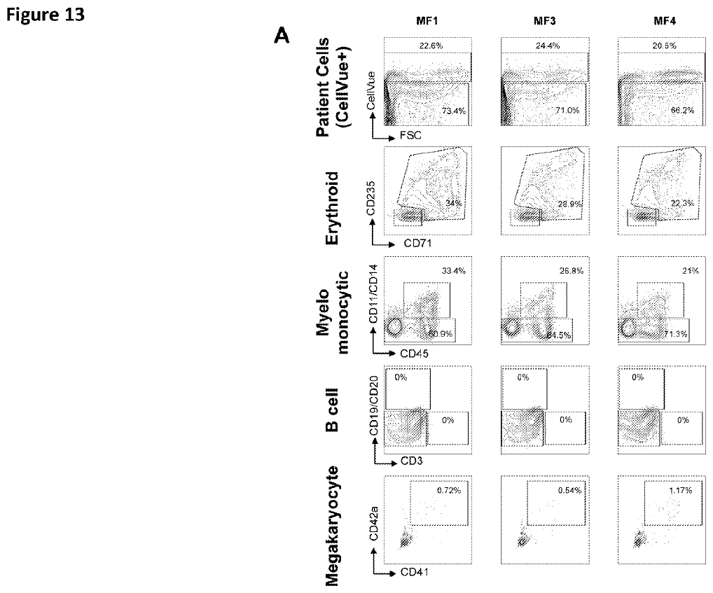

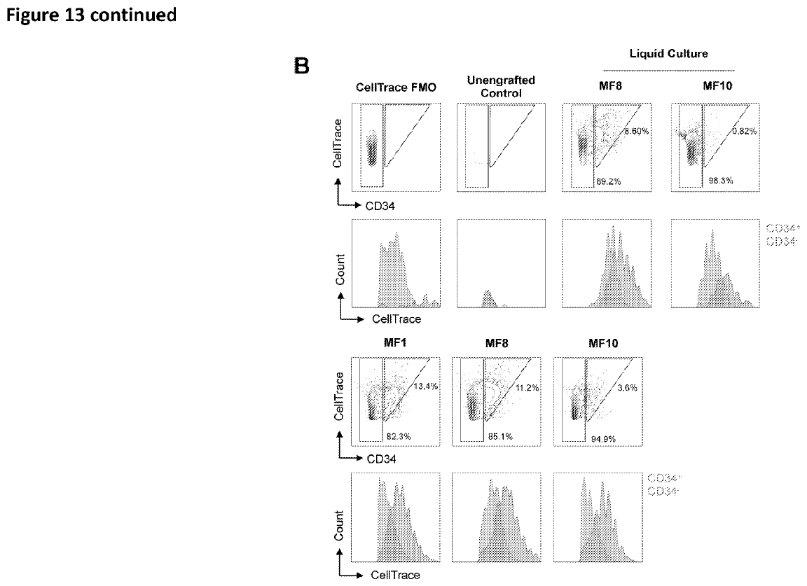

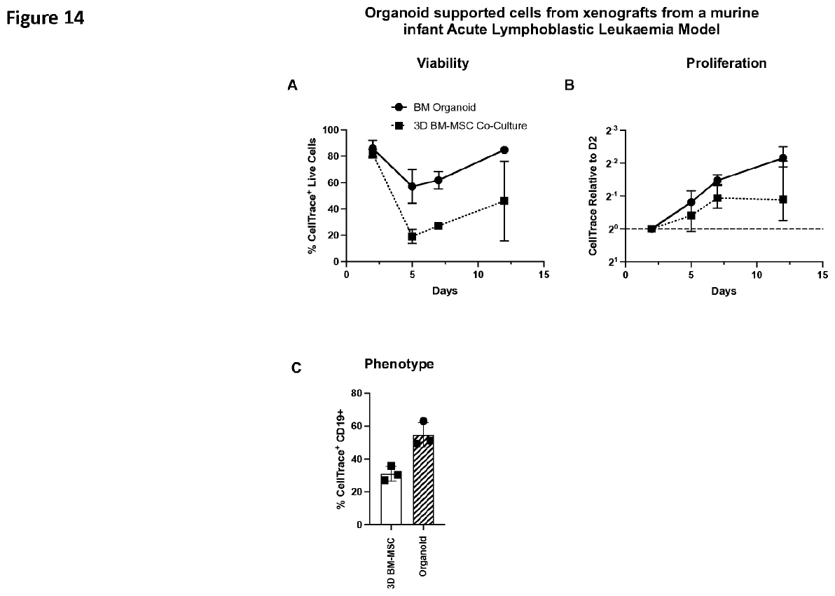

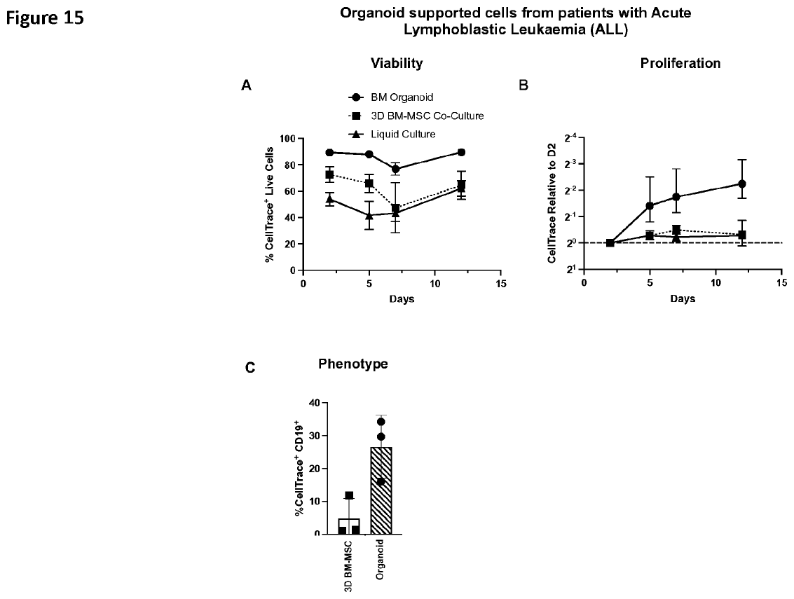

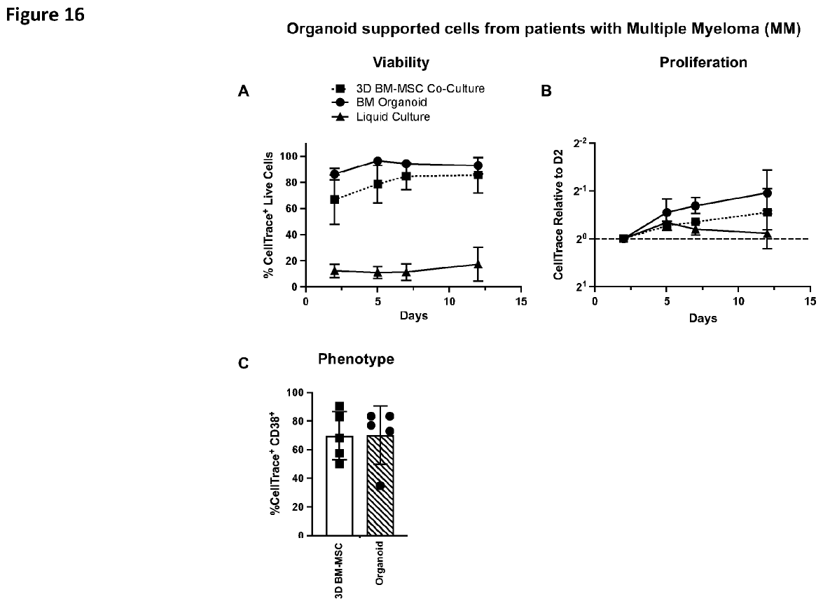

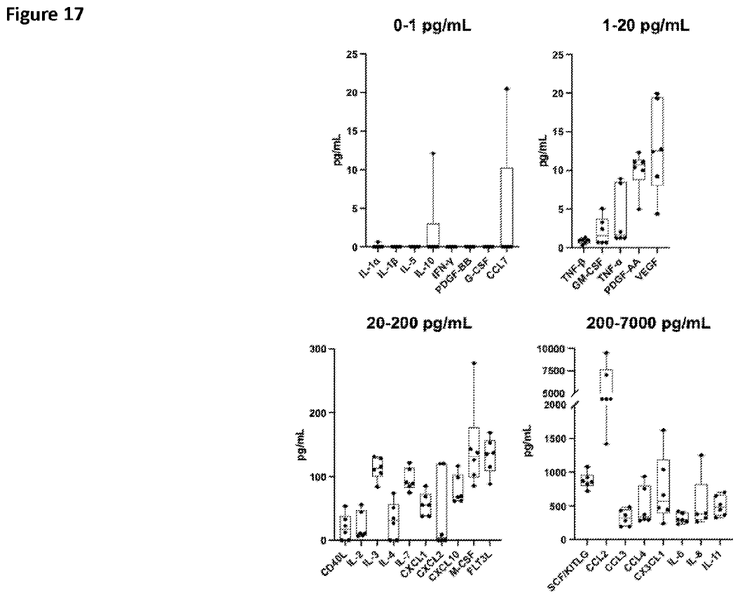

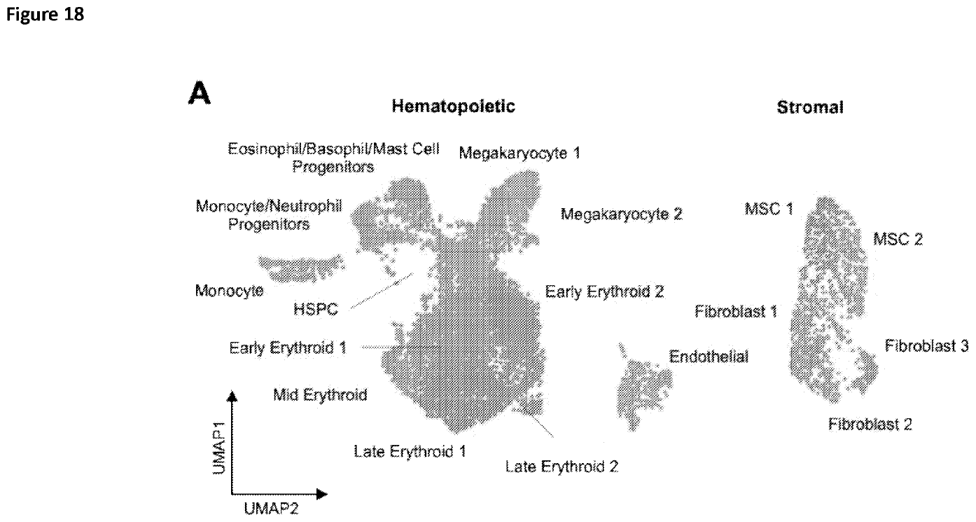

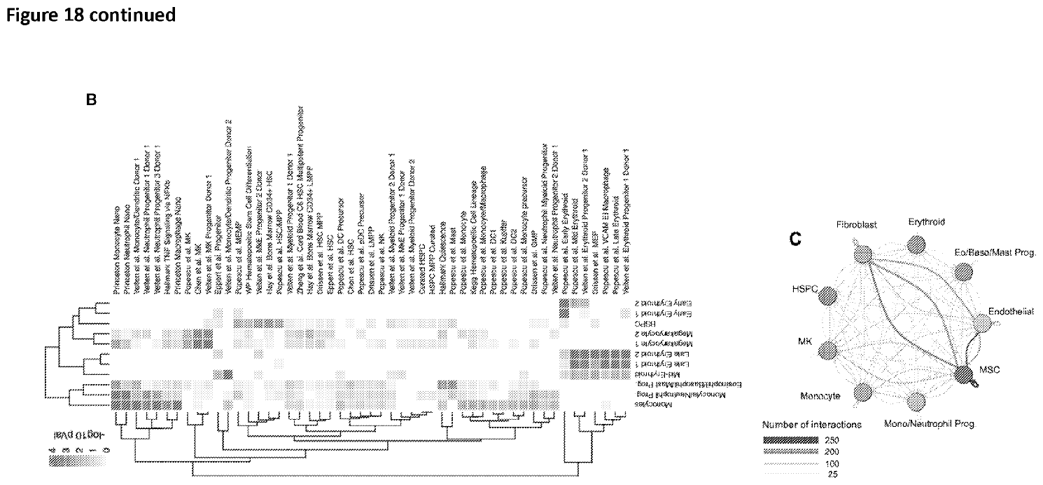

### iPSC Culture and Differentiation (for FIG. 11 Onwards)

A Gibco Human Episomal iPSC (Thermo Fisher Scientific Cat #A 18945) line was maintained in StemFlex medium (Thermo Fisher Scientific Cat #A3349401) and on Geltrex (Thermo Fisher Scientific Cat #A1569601)-coated 6-well plates. The iPSC line was karyotyped prior to use and potency markers assessed upon expansion and freezing. Cells were passaged as clumps using EDTA at 0.02% in PBS (0.5 mM, Sigma Cat #E8008), and were freshly thawed or passaged for differentiation and maintained in StemFlex supplemented with RevitaCell (Thermo Fisher Scientific Cat #2644501). Cultures were maintained at 37° C. and 5% CO₂. Differentiations were initiated with iPSCs between passages 5 and 30.

For differentiations, iPSC were dissociated using EDTA when colonies were approximately 100 μm in diameter. The resulting iPSC aggregates were incubated overnight in StemFlex supplemented with RevitaCell in 6-well Costar Ultra-Low Attachment plates (Corning Cat #3471) (day −1). After an overnight incubation, cells were collected by gravitation in a 15 mL Falcon tube (Fisher Scientific Cat #11507411) and resuspended in Phase I medium comprised of APEL2 (StemCell Technologies Cat #05275) supplemented with Bone Morphogenic Protein-4 (BMP4, Thermo Fisher Scientific Cat #PHC9531), Fibroblast Growth Factor-2 (FGF2, StemCell Technologies Cat #78134.1), Vascular Endothelial Growth Factor-A (VEGF-165, StemCell Technologies Cat #78159.1) at 50ng/mL, plated in a 6-well ULA plates and incubated at 5% O₂ for 3 days (d0-3).

Cell aggregates were then collected by gravitation and re-suspended in Phase II medium for a further 48 hours (d3-5). Phase II medium (APEL2 supplemented with BMP-4, FGF2, and VEGFA at 50 ng/ml; human Stem Cell Factor (hSCF, StemCell Technologies Cat #78062) and Fms-like tyrosine kinase-3 Ligand (Flt3, StemCell Technologies Cat #78009) at 25 ng/mL.

On d5 cells were collected by gravitation for hydrogel embedding. Hydrogels were composed of 60% collagen (either type I, type IV, or an equal parts type I+IV mix) and 40% Matrigel. Hydrogels were prepared on ice as per manufacturer's instructions and were comprised of Reduced Growth Factor Matrigel (Corning, Cat #354230) supplemented with 1 mg/mL human collagen type I (Advanced Biomatrix, Cat #5007) and human collagen type IV (Advanced Biomatrix, Cat #5022) as per designated gel composition. Hydrogel mixes were neutralised with 1N NaOH. An 0.5 mL cell-free base layer was added and allowed to polymerise for 2 hours, before a further 0.5 mL layer of gel supplemented with gravitated cell aggregates was added and also left to polymerise for 2 hours at 37° C. and 5% CO₂. Fully polymerised gels with cell aggregates were then supplemented with Phase III media comprised of VEGFA at either 50 ng or 25 ng/ml, VEGFC (where relevant) at 50 or 25 ng/mL, FGF2, BMP4, hSCF, Flt3, Erythropoeitin (EPO, StemCell Technologies, Cat #78007), Thrombopoeitin (TPO, StemCell Technologies, Cat #78210), Granuolocytic Colony-Stimulating Factor (G-CSF, StemCell Technologies, Cat #78012), at 25 ng/ml, and Interleukin-3 (IL3, StemCell Technologies, Cat #78194) and Interleukin-6 (IL6, StemCell Technologies, Cat #78050) at 10 ng/mL. Media was replenished every 72 hours.

### Immunofluorescence Staining

Sections were blocked using 2% Goat Serum (Thermo Fisher Scientific, Cat #31872) 1% Bovine Serum Albumin (BSA) (Sigma, Cat #A9418) prior to primary antibody labelling with antibody diluted in 1% BSA, sequential PBS washes, and finally secondary labelling with AlexaFluor conjugates. Whole organoid blocking solution was further supplemented with Triton X100, Tween, and Sodium deoxycholate.

Sprouting organoids were imaged within hydrogels in 8-well microslides (Ibidi, Cat #80806), whole organoids were labelled in 15 mL Falcons before embedding in 0.5% Agarose within 8-well microslides. Whole organoids were subject to serial dehydration (50%, 70%, 90%, 100%) within microslides before clearance with Ethyl Cinnamate and subsequent imaging. Sections were prepared by embedding fixed organoids in Optimal Cutting Temperature compound (OCT, VWR Cat #361603E) before sectioning onto Poly-L-Lysine covered slides. Slides were washed in Acetone before immunofluorescence labelling.

### Microscopy and Image Analysis

Confocal microscopy was performed using a Zeiss LSM880 confocal AiryScan microscope with either a 25×LD LCI plan apo 0.8 NA dual immersion (420852-9871-000) or 40×C-APO NA 1.2 water immersion objective (421767-9971-711) as described previously. Confocal images were acquired as representative Z-stacks (with Z-resolution set to Nyquist requirements), and presented as maximum intensity projections (Fiji) where stated. Histological preparations (reticulin and H&E, details provided in supplementary materials and methods) were imaged using a Zeiss AxioScan.Z1 slide scanner. Image analysis was performed in Fiji. For measurements of sprout radii, brightfield images acquired on an Evos (Thermo Fisher Scientific) desktop microscope. Sprout radii were measured manually by drawing and measuring a line from the centre to the tip of the sprout across 3 independent biological replicates, with between 30-50 sprouts measured per replicate. To measure the proximity of megakaryocytes to organoid blood vessels, 250 μm×50 μm volumes of individual organoids were acquired using cleared whole mount organoids imaged by confocal microscopy, as previously described. CD41 labelled megakaryocytes within 5 μm of UEAl labelled vessels were counted as ‘vessel-associated MKs’ within a maximum intensity projection of each imaged volume

### Single-Cell RNA-Sequencing

Cryopreserved cells pooled from 15 organoids from 3 differentiations from both VEGFA and VEGFA+C protocols were thawed, stained with DAPI to exclude non-viable cells, and DAPI-live cells sorted on a Becton Dickinson Aria Fusion with 100 nm nozzle as per recommendations in the 10× Genomics Single Cell Protocols—Cell Preparation Guide. 10,000 live cells per sample were sorted into 2 μL PBS/0.05% BSA (non-acetylated) and the cell number/volume adjusted to the target for loading onto the 10× Chromium Controller. Samples were processed according to the 10× protocol using the Chromium Single Cell 3′ library and Gel Bead Kits v3.1 (10× Genomics). Cells and reagents were prepared and loaded onto the chip and into the Chromium Controller for droplet generation. Reverse transcription was conducted in the droplets and cDNA recovered through demulsification and bead purification. Pre-amplified cDNA was used for library preparation, multiplexed and sequenced on a Novaseq 6000. Details on data processing ana

### scRNAseq Data Processing and Analysis

Demultiplexed FASTQ files were aligned to the human reference genome (GRCh38/hg38) using standard CellRanger (version 6.0.1) ‘cellranger count’ pipeline (10× Genomics). SingCellaR (https://supatt-lab.github.io/SingCellaR.Doc/) was used for the downstream analysis. Data was first subject to quality control with the maximum percentage of mitochondrial genes, maximum detected genes and max number of UMIs set to 12%, 6,000, and 50,000, respectively. Minimum detected genes and UMIs were set to 300 and 500, respectively and genes with minimum expressing cells was set as 10. Raw expression matrix was then normalised and scaled and number of UMIs and percentage of mitochondrial reads were regressed out before a general linear model (GLM) was used to identify highly variable genes were then subject to downstream analyses including principal component analysis (PCA), UMAP analysis (top 40 PCs were used, and n.neighbour=120), and clustering using the Louvain method. Differentially expressed genes were calculated using ‘identifyDifferentialGenes’ function (min.log2FC=0.3 and min.expFraction=0.25). To compare cells from the two experimental conditions (VEGFA only and VEGFA+C), cells were down-sampled so that each cell group had the same number of cells. Wilcoxon test of normalized UMIs was used to compare the gene expressions and Fisher's exact test was used to compare the cell frequency. The resulting P values from both tests were combined using Fisher's method and subsequently adjusted by Benjamini-Hochberg correction. ‘runFA2 ForceDirectedGraph’ function was used to identify the trajectories. CellPhoneDB v 2.1.1 (https://github.com/Teichlab/cellphonedb) was performed for ligand-receptor interactions using normalized expression matrix of VEGFA+C as detailed by Garcia-Alonso et al. Cell-cell interaction network between the different cell clusters from VEGFAC and Sankey plot demonstrating the interaction between TGFβ1, CXCL12, and CD44 ligands with their responding receptors from VEFGA and VEFGAC were plotted using a modified version of the CrossTalkeR R package (version 1.2.1).

We applied Symphony to map cells from VEGFA+C organoids to published scRNAseq datasets from human bone marrow and fetal liver and bone marrow cells respectively. For the human bone marrow dataset, we first built the reference data using the normalized expression matrix using ‘symphony::buildReference’. For the fetal liver dataset we used the pre-built reference provided by the Symphony developer. The ‘mapQuery’ and ‘knnPredict’ function were used to map the VEGFA+C cells onto the three reference datasets.

### Histology

Organoids were fixed in neutral buffered formalin (Sigma-Aldrich, Cat #HT501128-4L) in a 15 mL Falcon tube, washed twice with PBS, and then subject to serial dehydration (30%, 50%, 70%, 100%) in ethanol before immersion in Histoclear (Geneflow, Cat #A2-0101). Samples were then embedded in paraffin and sent as blocks to C&C laboratories for staining and mounting.

### CellTrace Labelling for Viability and Proliferation Assays

Primary cells were labelled with CellTrace Far Red as indicated by the manufacturer. Briefly, cells were washed 1× with PBS and resuspended at 1×10⁶ cells/mL in staining solution (CellTrace Far Red 2 μM in PBS). Cells were incubated in staining solution for 30 min at 37° C. After incubation CellTrace was quenched with 5 volumes of PBS with FBS (10%), spun down and resuspended in the appropriate media.

### Quantitative Real-Time Polymerase Chain Reaction (qRT-PCR)

Whole organoids were processed using either the Micro RNEasy Kit (Qiagen, Cat #74004) or Qiagen Mini RNA isolation kit (Qiagen, Cat #74104) according to the manufacturer's instructions. Isolated RNA was quantified on the NanoDrop ND-100 (Thermo Scientific) and cDNA was prepared using the High Capacity cDNA Reverse Transcription Kit (Applied Biosystems, Cat #4368814) or EvoScript Universal cDNA Master (Roche, Cat #07912374001) according to the manufacturer's instructions using standard cycling conditions. cDNA was diluted to 5 ng before being combined with PowerUp SYBR Green Master Mix reagent (Applied Biosystems, Cat #A25742) and the relevant PrimeTime qRT-PCR primers (IDT), or performed using TaqMan™ Universal PCR Master Mix (Applied Biosystems) on StepOne plus machine (Applied Biosystem) (see Suppl. Table 6 for a full list of primers). The absolute expression of the respective genes was calculated using the ΔCt method using GAPDH as an internal housekeeping control.

### Luminex Assays

To assess the production of growth factors, organoids were washed and cultured in StemPro-34 (L-Glutamine only) without any added supplements or growth factors for 12 days. 50:50 media changes were performed at 72 hour intervals, and media was collected for Luminex assays at day 12. Supernatant from 12 organoids was collected and pooled for each repeat.

Luminex kits (LXSHAM-03, LXSAHM-28) were used for multiplexed proteomic assays as per the manufacturer instructions. No detectable signal was observed in cell-free medium.

### Viability and Proliferation Assays

Primary cells were labelled with CellTrace Far Red as per kit instructions. Briefly, cells were washed and resuspended at 1×10⁶ cells/mL in staining solution for 30 min at 37° C. After incubation, CellTrace was quenched with 5 volumes of PBS with 10% FBS.

### Donor Cell Labelling

Prior to seeding, donor cells were labelled with CellVue Claret Far Red Fluorescent Cell Linker Mini Kit for General Membrane Labelling (Sigma Aldrich, Cat #MINCLARET-1KT) following manufacturer instructions. For seeding of organoids, each well of a 96-well plate containing 1-2 individual organoids or media alone were seeded with 5000 cells per well and cultured for up to 14 days in StemPro (Thermo Fisher Scientific, Cat #10639011) supplemented with Phase IV cytokines. Wells seeded with iALL cells were further supplemented with IL7, with 50% media changes every 2-3 days.

1. A method of generating bone marrow organoids from pluripotent stem cells wherein said method comprises:
a. driving induced pluripotent stem cells (iPSCs) to form mesodermal aggregates;
b. inducing vascular and hematopoietic commitment of the mesodermal aggregates by further culturing the mesodermal aggregates in medium, supplemented with recombinant BMP4, FGF2, VEGFA and SCF;
c. embedding the mesodermal aggregates in a hydrogel, and incubating the hydrogel in a sprouting medium comprising: a media suitable for stem cell maintenance supplemented with cytokines for differentiation, to form bone marrow organoids; and optionally
d. separating the bone marrow organoids from the hydrogel for further free culture.

2. The method of claim according to claim 1, wherein the media in step b. is further supplemented with Flt-3.

3. The method according to claim 1 or claim 2, wherein the mesodermal aggregates are formed by:
i. incubating induced pluripotent stem cells (iPSCs) to induce the formation of iPSC aggregates; and
ii. culturing the iPSC aggregates in a media for stem cell maintenance supplemented with BMP4, FGF2, and VEGFA, to induce formation of mesodermal aggregates.

4. The method according to any preceding claim, wherein the BMP4 is provided in the medium of step b. at a concentration of between about 10 and about 100 ng/ml.

5. The method according to any preceding claim, wherein the FGF2 is provided in the medium of step b. at a concentration of between about 10 and about 50 ng/ml.

6. The method according to any preceding claim, wherein the VEGFA is provided in the medium of step b. at a concentration of between about 10 and about 50 ng/ml.

7. The method according to any of claims 2-6, wherein the Flt-3 is provided in the medium of step b. at a concentration of between about 10 and about 100 ng/ml.

8. The method according to any preceding claim, wherein the SCF is provided in the medium of step b. at a concentration of about 1-100 ng/mL.

9. The method according to any preceding claim, wherein the vascular and haematopoietic commitment step comprises culturing the mesodermal aggregates for a period of between about 36 and 72 hours, or until the mesodermal aggregates achieve an average size of about 350-400 um and/or for a period until there are markers of early endothelial and haematopoeitic differentiation.

10. The method according to any preceding claim, wherein VEGFA is present in the sprouting medium at a concentration of between about 5 and 100 ng/ml.

11. The method according to any preceding claim, wherein FGF2 is present in the sprouting medium at a concentration of 10-100 ng/ml.

12. The method according to any preceding claim, wherein the sprouting medium comprises between about 1 and 20% Foetal Bovine Serum (FBS) or knock-out serum.

13. The method according to any preceding claim, wherein the sprouting medium comprises about 1-100 U/mL heparin sulfate.

14. The method according to any preceding claim, wherein the sprouting medium comprises about 1-50 ng/ml Interleukin 3 (IL3).

15. The method according to any preceding claim, wherein the sprouting medium comprises about 1-50 ng/ml Interleukin 6 (IL6).

16. The method according to any preceding claim, wherein the sprouting medium comprises about 1-100 ng/ml SCF.

17. The method according to any preceding claim, wherein the sprouting medium comprises about 1-100 ng/ml Flt3.

18. The method according to any preceding claim, wherein the sprouting medium comprises about 1-50 ng/ml thrombopoietin (TPO).

19. The method according to any preceding claim, wherein the sprouting medium comprises about 1-100 ng/ml Erythropoietin (EPO).

20. The method according to any preceding claim, wherein the sprouting medium comprises about 1-100 ng/ml Granulocyte Colony Stimulating Factor (G-CSF).

21. The method according to any preceding claim, wherein the sprouting medium comprises about 1-100 ng/ml BMP4.

22. The method according to any preceding claim, wherein the sprouting medium comprises VEGFC at a concentration of about 10-100 ng/ml.

23. The method according to any preceding claim, wherein the sprouting medium is changed during the maintenance period, and sprouting medium changes comprise a mixture of fresh and conditioned sprouting medium.

24. The method according to any preceding claim, wherein the hydrogel comprises collagen I and/or collagen IV.

25. Bone marrow organoids, wherein the bone marrow organoids are formed by the method according to any of claims 1-24.

26. Bone marrow organoids comprising haematopoietic stem cells (CD34+), monocyte and neutrophil cells (CD11b+), megakaryocytes (CD41+), erythroid cells (CD71+, CD235+), endothelium (CD31+, CD144+), and bone marrow mesenchymal stromal cells (PDGFRb+, LepR+, VCAM1+); optionally wherein the bone marrow organoid is formed by the method according to any of claims 1-24.

27. The bone marrow organoids according to claim 26, wherein the bone marrow organoids further comprise a lumen-forming vasculature network and/or sinusoidal endothelial cells.

28. A model for bone marrow fibrosis, wherein the model comprises bone marrow organoids according to any of claims 25-27 that have been treated with an agent to induce fibrosis and/or collagen deposition in the bone marrow organoids.

29. A method of producing a model for bone marrow fibrosis, wherein the method comprises treating bone marrow organoids according to any of claims 25-27 with an agent that is capable of inducing fibrosis and/or collagen deposition in the bone marrow organoids.

30. Use of the model for bone marrow fibrosis according to claim 28, or the method of claim 29, to identify agents capable of preventing or treating fibrosis, such as myelofibrosis, optionally wherein the bone marrow organoids are treated with a potential agent before, during or after the bone marrow organoids are treated to induce fibrosis, such as myelofibrosis.

31. A method of screening for agents capable of preventing or treating fibrosis, such as myelofibrosis, wherein bone marrow organoids according to any of claims 25-27 are treated with a potential agent before, during or after the bone marrow organoids are treated to induce fibrosis, such as myelofibrosis; and
A) determining if the potential agent has any effect in inhibiting or reducing the development of fibrosis, such as myelofibrosis, in the bone marrow organoids, or the reduction in fibrosis, such as myelofibrosis, after it has developed in the bone marrow organoids;
B) determining if the potential agent has any effect in inhibiting or reducing smooth muscle actin and/or collagen expression in the bone marrow organoids; and/or
C) determining if the potential agent has any effect in inhibiting or reducing collagen deposition in the bone marrow organoids.

32. A method for maintaining the viability of cells from a patient donor with blood cancer ex vivo, to enable mechanistic studies or screening of agents or potential agents consisting of one or more of:
A) determining if agent(s) or potential agent(s) have any effect in reducing the survival or proliferation of cancer cells from the patient;
B) determining if agent(s) or potential agent(s) have any effect on the pathogenicity of cancer cells from the patient;
C) determining if agent(s) or potential agent(s) have any effect on reducing the pathogenic remodelling of the organoid stroma or microenvironment/niche induced by cancer cells from the patient;
D) screening for potential biomarkers of cancer in the patient from whom engrafted cells are isolated;
E) studying clonal evolution of cancer in the organoids following seeding with cells from the patient, to predict future cancer progression in the patient donor; and
F) studying treatment response of cancer cells seeded in the organoids to determine the optimal treatments for the donor patients.

33. The use of the bone marrow organoids according to claims 25-27 to generate blood cells, for example for transfusion.

34. A composition comprising or consisting of two or more of, such as all of, recombinant BMP4, FGF2, VEGFA, Flt-3 and SCF.

35. The composition according to claim 34, wherein when recombinant BMP4 is provided in the composition for use at a concentration of between about 10 and about 100 ng/ml, recombinant FGF2 is provided for use at a concentration of between about 10 and about 50 ng/ml, recombinant VEGFA is provided for use at a concentration of between about 10 and about 50 ng/ml, recombinant Flt-3 is provided for use at a concentration of between about 10 and about 100 ng/ml, and/or recombinant SCF is provided for use at a concentration of between about 1 and about 100 ng/ml.

36. A method of producing hematopoietic and/or stromal cells, wherein said method comprises:
a. driving induced pluripotent stem cells (iPSCs) to form mesodermal aggregates;
b inducing vascular and haematopoietic commitment of the mesodermal aggregates by further culturing the mesodermal aggregates in medium, supplemented with recombinant BMP4, FGF2, VEGFA, Flt-3 and SCF;
c. embedding the mesodermal aggregates in a hydrogel, and incubating the hydrogel in a sprouting medium comprising: a media suitable for stem cell maintenance supplemented with cytokines for differentiation, to form a mixture of hematopoietic and stromal cells.

37. Hematopoietic and/or stromal cells, which are obtained by the method of claim 36.

38. A kit comprising one or more, such as all of, recombinant BMP4, FGF2, VEGFA, Flt-3 and SCF.

39. The kit of claim 38, wherein the one or more of the recombinant BMP4, FGF2, VEGFA, Flt-3 and SCF are provided in one solution in the kit, or two, three, four of five separate solutions in the kit.

40. The kit of claim 38 or 39, further comprising a mesoderm-inducing medium, sprouting medium and/or hydrogel for carrying out the method of any of claims 1-25, 29, 31 and 36.
